(19) United States
(12) Patent Application Publication
(10) Pub. No.: US 2018/0000737 A1
(43) Pub. Date: Jan. 4, 2018

(54) INJECTABLE AGGREGATES FOR JOINT AND SOFT TISSUE DISTRESS

(71) Applicant: VISGO THERAPEUTICS, INC., Bedford, MA (US)

(72) Inventors: Jeanne M. Bertonis, Arlington, MA (US); Paul Higham, Ringwood, NJ (US); Pamela A. Hay, Bedford, MA (US)

(73) Assignee: VISGO THERAPEUTICS, INC., Bedford, MA (US)

(21) Appl. No.: 15/544,851

(22) PCT Filed: Jan. 19, 2016
(86) PCT No.: PCT/US16/13958

§ 371 Date: Jul. 19, 2017

Related U.S. Application Data
(60) Provisional application No. 62/105,703, filed on Jan. 20, 2015.
(60) Provisional application No. 62/271,675, filed on Dec. 28, 2015.

(51) Int. Cl. A61K 9/14; A61K 31/196; A61K 9/00

(52) U.S. Cl. CPC A61K 9/146; A61K 9/0019; A61K 31/196

(57) ABSTRACT

Aggregates formed from one or both of chitosan and hyaluronan, suspended or otherwise dispersed in a liquid carrier are useful for treating joint or muscle distress in a subject, where the composition may include one or more pharmaceutically active agents and the composition in locally delivered to the joint or muscle by, for example, injection.

### CROSS-REFERENCE TO RELATED APPLICATIONS

This application claims the benefit under 35 U.S.C. §119(e) of U.S. Provisional Patent Application No. 62/105,703 filed Jan. 20, 2015, and U.S. Provisional Patent Application No. 62/271,675 filed Dec. 28, 2015, which applications are incorporated herein by reference in their entireties for all purposes.

### FIELD OF THE INVENTION

The present invention relates generally to pharmaceutical products and their use, and more particularly to injectable compositions comprising polymer(s) and pharmaceutically active agent(s), for use in, for example, treating joint disorders such as osteoarthritis and rheumatoid arthritis.

### SUMMARY

Briefly, in one embodiment, the present disclosure provides a composition comprising one or more aggregates. In one aspect, the aggregates comprise hyaluronan, chitosan and a pharmaceutical agent. In other aspects, the composition includes aggregates that comprise only one or two of hyaluronan, chitosan and pharmaceutical agent. In one aspect, the composition is in a dry form, which is suitable for storage and/or direct delivery to a patient. In other aspects the composition includes a liquid carrier, e.g., water.

In yet other embodiments, the present disclosure provides methods for treating a patient in need thereof. For example, a composition of the present disclosure may be injected into the joint of a patient having joint distress, in order to achieve beneficial results such as pain alleviation and/or increased lubrication of the joint and/or inhibition of the processes that underlie certain forms of arthritis. As another example, a composition of the present disclosure may be injected into the joint of a patient having joint distress, in order to achieve a structural healing or repair of the underlying condition that is giving rise to the distress. Exemplary joints include a knee, hip, elbow, wrist, ankle, shoulder, finger or toe joint. The joint may be any one or more of the carpometacarpal (CMC) joints, which are five joints in the wrist. As a third example, a composition of the present disclosure may be injected into soft tissue, e.g., soft tissue that is damaged or inflamed, e.g., the soft tissue giving rise to the condition known as tennis elbow.

For example, in one embodiment the present disclosure provides a composition comprising aggregates, which may also be referred to as particles, where the particles comprise crosslinked chitosan having amino (—NH₂) groups. The particles also comprise an active pharmaceutical ingredient (API) that is incorporated into the particles. The particles, which may be spherical or non-spherical, may be characterized by a diameter, the particles having a diameter of greater than about 20 microns, e.g., greater than 21, or 22, or 23, or 24, or 25, or 26, or 27, or 28, or 29 or 30 microns and less than about 100 microns, e.g., less than 110 microns, or 105, or 100, or 95, or 90 microns. The crosslinked chitosan is the reaction product of a crosslinking agent and a chitosan, where the chitosan has a degree of deacetylation (DDA) of greater than 75% and less than 95% or ranges with values therein, e.g., greater than 80% and less than 95%. The API may be, for example, an NSAID, where an exemplary NSAID is diclofenac in salt or acid form. Optionally, at least some of the diclofenac is present in a crystalline form. The crosslinked chitosan is the reaction product of chitosan and a crosslinking agent, where the crosslinking agent is optionally selected from those crosslinking agents that covalently react with amino groups. For example, in one embodiment, the crosslinked chitosan is the reaction product of glutaraldehyde and chitosan which provides for covalent crosslinks. The crosslinked chitosan is the reaction product of a crosslinking agent and a chitosan, where the chitosan may be characterized by its intrinsic viscosity. The intrinsic viscosity of the chitosan is between 1 and 1,000 mPas, and may optionally be selected from chitosans having an intrinsic viscosity of greater than 50 mPas and less than 300 mPas. The composition may include a liquid medium so that the composition is injectable into a subject in need of the API, through a needle of 18-27 gauge. Thus, the present disclosure provides, in one embodiment, a composition comprising particles, the particles comprising crosslinked chitosan having amino (—NH₂) groups, and an active pharmaceutical ingredient (API) incorporated into the particles, the particles having a diameter of greater than 25 microns and less than 100 microns, wherein the crosslinked chitosan is the reaction product of a crosslinking agent and a chitosan having a degree of deacetylation (DDA) of greater than 75% and less than 95%, wherein the API is diclofenac and at least some of the diclofenac is in crystalline form, the crosslinked chitosan is the reaction product of glutaraldehyde and chitosan which provides for covalent crosslinks, and the crosslinked chitosan is the reaction product of a crosslinking agent and a chitosan having an intrinsic viscosity of greater than 50 mPas and less than 300 mPas.

In another embodiment, the present disclosure provides a method of delivering a particle into a synovial fluid and a synovium of a subject to provide a depot for the sustained release of an active pharmaceutical agent (API) from the particle. The method comprises providing a liquid composition comprising particles according to any of the embodiments disclosed herein, e.g., particles comprising crosslinked chitosan having amino (—NH₂) groups, and an active pharmaceutical agent (API) incorporated into the particles, where the particles have a diameter of greater than 25 microns and less than 100 microns, and wherein the crosslinked chitosan is the reaction product of a crosslinking agent and a chitosan having a degree of deacetylation (DDA) of greater than 75% and less than 95%. The method also includes providing a syringe containing the liquid composition, where the syringe has a needle in the range of 18-27 gauge. The syringe is used to intra-articularly inject the liquid composition through the needle and into a subject in need thereof. The particles are too large to be engulfed by macrophages, but due to the amino groups present on the particles, the particles will interact with and become embedded within the synovium, i.e., they will reside amongst the cells of the synovium of the subject. The particles may release API directly into the surrounding synovium and provide a sustained release of the API, e.g., a sustained release of greater than 24 hours. The particles may optionally delivery a burst of API immediately upon injection into the subject, and thereafter release the API over an extended period of time of at least 24 hours. Optionally, the API is diclofenac, and at least some of the diclofenac is incorporated into the particle in crystalline form.

The details of one or more embodiments are set forth in the description below. The features illustrated or described in connection with one exemplary embodiment may be combined with the features of other embodiments. Other features, objects and advantages will be apparent from the description, the drawings, and the claims. In addition, the disclosures of all patents and patent applications referenced herein are incorporated by reference in their entirety.

### BRIEF DESCRIPTION OF THE DRAWINGS

Exemplary features of the present disclosure, its nature and various advantages will be apparent from the accompanying Figures and the following detailed description of various embodiments.

FIG. 1 provides four pictures of sheep synovial membrane, labeled 1A, 1B, 1C and 1D, in which chitosan aggregates with diclofenac active agent according to the present disclosure are embedded.

FIG. 2 provides a picture of sheep synovial membrane in which chitosan aggregates with diclofenac (some of which are labeled with the number 1) are shown in the vicinity of, but not inside of, synovium macrophages (some of which are labeled with the number 2).

### DETAILED DESCRIPTION OF THE INVENTION

As one embodiment, the present disclosure provides a composition comprising aggregates distributed in a liquid carrier. In one aspect, the aggregates comprise chitosan and crosslinked chitosan, and an active pharmaceutical ingredient (API). In one aspect, the aggregates comprise hyaluronan, chitosan and a pharmaceutical agent. The carrier may comprise water, and may optionally include components such as salt(s) and additional pharmaceutical agent(s). The compositions, the various components of the compositions, and methods of preparing and using the compositions will be described in the following paragraphs. In addition, the present disclosure makes use of various terms and abbreviations which will be defined below as an aid to the reader's understanding of the disclosure.

### Definitions and Conventions

Aggregate refer to the result of the gathering together of one or more chemical species, such as polymer chains or pharmaceutical agents, into an adhesive or cohesive unit. The components of an aggregate associate with one another and create an individual unit which is distinct from a neighboring aggregation of chemical species. While the term aggregate will be used frequently herein, the terms mass or particle or assemblage or granule or cluster or bead or sphere or microsphere or microcapsule or microparticle are alternative terms that may be substituted for the term “aggregate” at any occurrence herein.

CA refers to aggregates formed from chitosan. CA do not contain hyaluronan or pharmaceutical agent, however may contain other components.

CDA refers to drug loaded chitosan-containing aggregates. CDA do not contain hyaluronan, however may contain other components.

CHA refers to aggregates formed from both chitosan and hyaluronan. CHA do not contain any pharmaceutical agent, however may contain other components.

CHDA refer to drug loaded aggregates formed from both chitosan and hyaluronan. CHDA may contain other components.

DA refers to aggregates formed from pharmaceutical agent (drug). DA do not contain either chitosan or hyaluronan, but may contain other components.

HA refers to aggregates formed from hyaluronan. HA do not contain either chitosan or pharmaceutical agent, however may contain other components.

HDA refers to drug loaded hyaluronan-containing aggregates. HDA do not contain chitosan, however may contain other components.

nAC refers to non-aggregated chitosan, or in other words, chitosan that is not part of an aggregate with itself or any other material, but instead is dissolved or otherwise dispersed in the carrier.

nAD refers to non-aggregated pharmaceutical agent (drug), or in other words, drug that is not part of an aggregate with itself or any other material, but instead is dissolved or otherwise dispersed in the carrier.

nAH refers to non-aggregated hyaluronan, or in other words, hyaluronan that is not part of an aggregate with itself or any other material, but instead is dissolved or otherwise dispersed in the carrier.

Both the aggregates and the compositions of the present disclosure may include more than one drug. When a single aggregate contains more than one drug, the aggregate may be abbreviated using the convention d1, d2, d3, etc. to refer to a first drug, a second drug, a third drug, etc., in lieu of the “D” in the naming of the aggregate. For example, a CDA having two different drugs may be referred to as Cd1d2A. Likewise, a CHDA having three different drugs may be referred to as CHd1d2d3A. When a composition contains more than one drug, the component names can indicate the presence of a plurality of drugs. For example, if the composition contains CDA and HDA, and the drug in the CDA is different from the drug in the HDA, then optionally, the composition may be described as containing Cd1A and Hd2A. If the d1, d2 etc. nomenclature is not specified, then the “D” may optionally be replaced with d1, d2 etc. as needed to denote a plurality of drugs in an aggregate, and likewise with a plurality of drugs in a composition.

Subject refers to a person or animal in need of treatment. For example, a person or animal (e.g., a dog, cat, horse) that has joint distress such as joint pain, joint inflammation, degradation of cartilage in a joint, osteoarthritis, and/or rheumatoid arthritis is a person in need of treatment.

### Aggregate Components

The aggregates of the present disclosure may contain hyaluronan. As used herein, the terms “hyaluronic acid,” and “hyaluronan,” and “HA” are used interchangeably to refer to hyaluronic acids or salts of hyaluronic acid, such as the sodium, potassium, magnesium, and calcium salts, among others. Hyaluronan is a glycosaminoglycan (GAG), and in particular hyaluronan is an unbranched polysaccharide made up of alternating glucuronic acid and N-acetyl glucosamine units. It is a viscoelastic material that is found in the extracellular matrix of cartilage. Hyaluronan is an important building component of aggregated proteoglycans which impart resilient characteristics of articular cartilage. Hyaluronan not only helps keep the cartilage that cushions joints strong and flexible, but it also helps increase supplies of joint-lubricating synovial fluid.

Thus, in one embodiment, the present disclosure provides a composition comprising particles suspended in a liquid carrier, the particles comprising at least one of chitosan and crosslinked chitosan; the liquid carrier comprising hyaluronic acid and at least one of buffer and water. Optionally, the particles incorporate an active pharmaceutical agent (API), and the particles provide extended release of the API into a subject in need thereof that receives the composition by administration. Also optionally, the hyaluronic acid provides visco-supplementation to a joint of a subject that receives the composition by administration.

In addition, an embodiment of the present disclosure provides particles comprising hyaluronic acid and at least one of chitosan and crosslinked chitosan. In one embodiment the particles comprise crosslinked chitosan. Optionally, the particles further comprise an active pharmaceutical agent (API), where the API may be an NSAID such as diclofenac, where optionally at least some of the diclofenac is in crystalline form.

Due to the presence of the glucuronic acid residues, and in particular due to the carboxylic acid groups on the glucuronic acid residues, hyaluronan is an acidic material. Accordingly, hyaluronan is soluble in mildly alkaline water, and will form ionic complexes, i.e., salts, with positively charged species, e.g., chitosan.

The hyaluronan used in the present disclosure includes natural formulations, synthetic formulations, or combinations thereof. The hyaluronan can be provided in liquid or solid formulations, and the hyaluronan can be in pure liquid form or in a solvent at various concentrations.

The hyaluronan may be physically or chemically modified in order to render it more suitable for use in the present compositions and methods. Examples of chemical modifications which may be made to the HA include any reaction with one or more of the four reactive groups of HA, namely the acetamido, carboxyl, hydroxyl, and the reducing end.

Since hyaluronan is a polymeric molecule, it can be characterized by a number average or weight average molecular weight. Hyaluronan suitable for use according to the present disclosure can have various molecular weights. Suitable molecular weights include low molecular weight (“LWM”) hyaluronan of about 400 to 500 kilodaltons (kDa), medium molecular weight (“MMW”) hyaluronan of about 500-1000 kDa, and high molecular weight (“HMW”) hyaluronan of about 1.0-4.0 million daltons (MDa). In various optional embodiments, the hyaluronan has a molecular weight of 150 KDa to 1.8 MDa. In various embodiments, the molecular weight can be, for example, 400, 500, 600, 700, 800, 900, 1000, 1100, 1200, 1300, 1400, 1500 KDa or more, or any range derivable therein. Two exemplary hyaluronans are sodium hyaluronate from Lifecore Biomedical (Chaska, Minn.) one having an average molecular weight, viscosity (Mw) of about 730 KDa and another having an average molecular weight, viscosity(Mw) of 1350 KDa which come from the supplier as a white powder or fibrous aggregate.

Chemically modified or crosslinked hyaluronan may have very different molecular weights than described above. Regardless, these materials are also applicable in this invention preferably when combined with a non-crosslinked or lightly crosslinked HA in the formulation.

Suitable sources for hyaluronan include Lifecore Biomedical (Chaska, Minn.) and Novozymes Biopharma US Inc. (Cambridge, Mass.).

The aggregates of the present disclosure may contain chitosan. Chitosan is typically derived from chitin, where chitin is found as a basic component of crustacean shells such as obtained from shrimp and crab, among other sources. Chemically, chitin is a linear polymer of β-(1,4)-linked N-acetyl-glucosamine residues. The deacetylation of chitin produces chitosan, where chitosan is a copolymer of randomly distributed D-glucosamine (i.e., deacetylated N-acetyl-glucosamine) and N-acetyl-glucosamine residues. Thus, the deacetylation reaction of chitosan converts an N-acetyl group to an amino group.

Due to the presence of amino groups, chitosan is a weak base with a pKa value of the glucosamine residues being about 6.2-7.0. Chitosan is therefore insoluble at neutral and alkaline pH. However in acidic media, the amino groups of chitosan as well as crosslinked chitosan are protonated, resulting in a soluble, positively charged polysaccharide, i.e., a salt of chitosan.

The extent to which chitosan may carry a positive charge depends on the degree to which deacetylation occurs during its formation. Chitosan manufacturers measure and report a degree of deacetylation (DDA) for the various chitosans they produce and sell. The chitosan of the present disclosure typically has a DDA of greater than 60%, or greater than 70%, or greater than 80%, or greater than 85%, or greater than 90%, or greater than 92%, or greater than 94%. The chitosan may also be described in terms of the maximum DDA, where in various embodiments the chitosan DDA is less than 100% or less than 95% or is less than 93%. For example, a suitable chitosan may have a DDA of about 80-95%, while another suitable chitosan may have a DDA of 85-95%. Other suitable DDA ranges for the particles of the present disclosure are provided elsewhere herein.

The chitosan may be characterized in terms of its molecular weight (MW). That molecular weight may be expressed in terms of Daltons (g/mol). Sometimes, and particularly for very high molecular weight chitosan, manufacturers provide a solution viscosity measurement as an indicator of molecular weight. The chitosan used in the present compositions will typically have a range of molecular weight, where exemplary ranges may be selected from 10 k-50 k, 30 k-80 k, 60 -150 k, 110-150 k, 150-250 k, 150-350 k, 150-500 k, and 250-600 k g/mol, where molecular weight may be measured on the basis of the molecular weight of the corresponding chitosan acetate. When apparent viscosity values are being used to characterize molecular weight, suitable viscosity values are from about 1 to about 1,000 mPas, or from about 10 to about 1,000 mPa-s. Other suitable intrinsic viscosity ranges for the particles of the present disclosure are provided elsewhere herein.

Both chitin and chitosan are readily available from many suppliers. Indeed, after cellulose, chitin is the most-abundant polysaccharide found on Earth, and is available from both crustacean and fungal sources, to name two sources. Suitable suppliers of chitosan include, but are not limited to, NovaMatrix (a business unit of FMC BioPolymer, Philadelphia, Pa.), Heppe Biomaterial GmbH (Landsberg, Germany), Altakitin Corp. (Toronto, Calif.) and Mycodev Group, (New Brunswick, Canada). Each of these companies offers chitosan products for sale which may be used in the compositions of the present disclosure.

The method of sterilizing the composition of the present disclosure may change the molecular weight of the component(s). For example, if ionizing radiation is used to sterilize the composition, then the molecular weight of the polymers of the non-sterile composition may need to be greater than the targeted molecular weight of the polymers of the sterile composition, since ionizing radiation can cause polymer chain scission and thus a decrease in molecular weight. The molecular weight of the chitosan may be greater than 250 kg/mol in those instances where it is envisioned that ionizing radiation will be used for sterilization.

The aggregates of the present disclosure may contain a pharmaceutical agent. Alternatively, or additionally, a pharmaceutical agent may be dissolved or suspended in the carrier. The pharmaceutical agent may control symptoms, e.g., reduce pain, or it may prevent disease progression, e.g., interfere with the progression of cartilage damage. The compositions of the present disclosure may include more than one pharmaceutical agent, e.g., the composition may include a first agent that reduces pain and a second agent that prevents disease progression and/or improves joint health. The pharmaceutical agent may be referred to herein as the drug, or as the active pharmaceutical ingredient (API), where these terms are used synonymously herein.

In one aspect, the pharmaceutical agent is a pain medication, also known as an analgesic. A suitable class of analgesic for the present disclosure is an opioid. Suitable opioids may be selected from Paracetamol/Acetaminophen, Dextropropoxyphene, Codeine, Tramadol, Tapentadol, Anileridine, Alphaprodine, Pethidine, Hydrocodone, Morphine, Oxycodone, Methadone, Diamorphine, Hydromorphone, Oxymorphone, Levorphanol, 7-Hydroxymitragynine, Buprenorphine, Fentanyl, Sufentanil, Bromadol, Etorphine, Dihydroetorphine, Carfentanil, and Heroin.

These particular opioids, which tend to cause addiction, are known as narcotic opioids. As mentioned previously, the aggregates as disclosed herein may contain chitosan. One benefit of introducing chitosan to a subject having joint distress is that chitosan has been shown to have an analgesic effect on inflammatory pain (see, e.g., Okamoto Y., et al., Carbohydrate Polymers 49:249-252 (2002)), perhaps by absorbing excess protons. Thus, chitosan provides not only a carrier function for hyaluronan and/or pharmaceutical agent, but it also may provide the benefit of reducing pain associated with joint distress. In part, this may be due to the fact that chitosan breaks down, in vivo, to glucosamine, which is a DMOAD discussed later herein. Thus, in one embodiment the present disclosure provides a method of delivering glucosamine to a subject in need thereof, the method comprising administering a composition comprising chitosan to a joint of the subject in need of glucosamine. In additional embodiments, any one or more of the following exemplary features may further characterize the method of delivering glucosamine to a subject in need thereof: the glucosamine is delivered over an extended time period by degradation of the chitosan; the chitosan is degradable by lysozyme to provide an extended delivery of glucosamine to the subject; a single injection of chitosan provides an extended delivery of glucosamine to the subject at a non-toxic concentration; a single injection of chitosan provides an extended delivery of glucosamine to the subject at an analgesic concentration; a single injection of chitosan provides an extended delivery of glucosamine to the subject at a non-inflammatory concentration; and a single injection of chitosan provides a disease-modifying amount of glucosamine to the joint.

In one aspect, the pharmaceutical agent is a steroid, i.e., a corticosteroid. Examples of corticosteroid medications include triamcinolone, cortisone, prednisone, dexamethasone and methylprednisolone. Low doses of steroids may provide significant relief from pain and stiffness for people with conditions including rheumatoid arthritis. Temporary use of higher doses of steroids may help a person recover from a severe flare-up of arthritis. Steroids when taken orally or intravenously can have many side effects. By delivering the steroid directly to the joint space according to the present disclosure, the risk of these side-effects is greatly reduced. In one aspect, the pharmaceutical agent is a non-steroidal anti-inflammatory drug (NSAID). Exemplary NSAIDs include, without limitation (common trade name(s) for a named NSAID are provided parenthetically following the generic name for the NSAID): aspirin (Anacin, Ascriptin, Bayer, Bufferin, Ecotrin, Excedrin), choline and magnesium salicylates (CMT, Tricosal, Trilisate), choline salicylate (Arthropan), celecoxib (Celebrex), diclofenac potassium (Cataflam), diclofenac sodium (Voltaren, Voltaren XR), diclofenac sodium with misoprostol (Arthrotec), diflunisal (Dolobid), etodolac (Lodine, Lodine XL), fenoprofen calcium (Nalfon), flurbiprofen (Ansaid), ibuprofen (Advil, Motrin, Motrin IB, Nuprin), indomethacin (Indocin, Indocin SR), ketoprofen (Actron, Orudis, Orudis KT, Oruvail), magnesium salicylate (Arthritab, Bayer Select, Doan's Pills, Magan, Mobidin, Mobogesic), meclofenamate sodium (Meclomen), mefenamic acid (Ponstel), meloxicam (Mobic), nabumetone (Relafen), naproxen (Naprosyn, Naprelan), naproxen sodium (Aleve, Anaprox), oxaprozin (Daypro), piroxicam (Feldene), rofecoxib (Vioxx), salsalate (Amigesic, Anaflex 750, Disalcid, Marthritic, Mono-Gesic, Salflex, Salsitab), sodium salicylate, sulindac (Clinoril), tolmetin sodium (Tolectin), and valdecoxib (Bextra).

In one aspect, the present disclosure provides formulations comprising NSAID and chitosan. In another aspect, the present disclosure provides formulations comprising NSAID and hyaluronic acid. In another aspect, the present disclosure provides formulations comprising NSAID, chitosan and hyaluronic acid. As mentioned previously, the aggregate may be in the form of a microsphere. As discussed below, the NSAID may inhibit cyclooxygenase, and hence, prostaglandin synthesis.

In one aspect, the pharmaceutical agent is diclofenac, which is also known by several trade names including Aclonac, Cataflam, and Voltaren. Under IUPAC nomenclature, diclofenac is known as 2-(2-(2,6-dichlorophenylamino)phenyl)acetic acid. The exact biological mechanism of action for diclofenac is not entirely known. The primary mechanism responsible for its anti-inflammatory, antipyretic, and analgesic action is thought to be inhibition of prostaglandin synthesis by inhibition of cyclooxygenase (COX). Research suggests diclofenac can inhibit the thromboxane-prostanoid receptor, affect arachidonic acid release and uptake, inhibit lipoxygenase enzymes, and activate the nitric oxide-cGMP antinociceptive pathway. Other novel MOAs may include the inhibition of substrate P, inhibition of peroxisome proliferator activated receptor gamma (PPARgamma), blockage of acid-sensing ion channels, alteration of interleukin-6 production, and inhibition of N-methyl-D-aspartate (NMDA) receptor hyperalgesia. In one aspect, the present invention provides compositions and methods that include diclofenac. Benefits of diclofenac are disclosed in, for example, Gan, T. J., Current Medical Research & Opinion, 26(7):1715-1731 (2010).

In another aspect, the pharmaceutical agent is indomethacin, which under IUPAC nomenclature is named 2-{1-[(4-chlorophenyl)carbonyl]-5-methoxy-2-methyl-1H-indol-3-yl}lacetic acid. This NSAID is also known as indometacin, along with many other trade names. Indomethacin is a nonselective inhibitor of cyclooxygenase (COX) 1 and 2, enzymes that participate in prostaglandin synthesis from arachidonic acid. Indomethacin has two additional modes of actions with clinical importance, namely: i) inhibition of the motility of polymorphonuclear leukocytes, similar to colchicine; and ii) uncoupling of oxidative phosphorylation in cartilaginous (and hepatic) mitochondria, like salicylates. Taken together, these functions allow indomethacin to have both analgesic and the anti-inflammatory properties. In one aspect, the present invention provides compositions and methods that include indomethacin.

In another aspect, the pharmaceutical agent is ketoprofen, which under IUPAC nomenclature is named (RS)-2-(3-benzoylphenyl)propanoic acid. Ketoprofen is used for its antipyretic, analgesic, and anti-inflammatory properties by inhibiting cyclooxygenase-1 and -2 (COX-1 and COX-2) enzymes reversibly, which decreases production of proinflammatory prostaglandin precursors. In one aspect, the present invention provides compositions and methods that include ketoprofen.

In another aspect, the pharmaceutical agent is ibuprofen, which under IUPAC nomenclature is named RS)-2-(4-(2-methylpropyl)phenyl)propanoic acid. Ibuprofen functions by inhibiting the enzyme cyclooxygenase (COX), which converts arachidonic acid to prostaglandin H2 (PGH2). PGH2,in turn, is converted by other enzymes to several other prostaglandins which are mediators of pain, inflammation, and fever. Ibuprofen is a common treatment for rheumatoid arthritis, and may be included in any of the compositions and methods of the present disclosure.

In one aspect, the pharmaceutical agent is a disease-modifying antirheumatic drug, commonly abbreviated as DMARD. DMARDs include hydroxychloroquine (Plaquenil), Leflunomide (Arava), Cyclosporine (Neoral), Sulfasalzine (Azulfidine), Methotrexate (Rheumatrex, Trexall), Azathioprine (Imuran), Cyclophosphamide (Cytoxan), and various biologics (Actemra, Cimzia, Enbrel, Humira, Kineret, Orencia, Remicade, Rituxan, Simponi).

In one aspect, the pharmaceutical agent is a disease modifying osteoarthritis (OA) drug, commonly abbreviated as DMOAD. DMOADs reduce or stop the progression of osteoarthritis by, for example, targeting cartilage catabolism and/or anabolism, targeting synovial membrane inflammation, or targeting subchondral bone remodeling, such as targeting a reduction in abnormal subchondral bone metabolism activities via preventing the resorption of this tissue and/or increasing the mineralization. As another example, DMOADs may decrease the levels and/or activity of matrix metalloproteinase (MMP) enzymes and/or adamalysin (ADAMTS) enzymes, both of which are implicated in the depletion of proteoglycans and a breakdown of the collagen network in arthritic joints. DMOADs may inhibit proteases (e.g., collagenases and gelatinases) and/or cytokines (e.g., IL-1β, IL-6, or TNF-α) in the joint space. DMOADs may inhibit pro-inflammatory cytokine-induced signalling pathways by, for example, targeting adenosine 3 receptor, MAP kinase (e.g., p38α) or IKK. See, e.g., Martel-Pelleteir J., et al. Bone 51:297-311 (2012).

DMOADs suitable for use in the present disclosure include chondroitin sulfate (e.g., see Institut Biochimique Societe Anonyme sponsored clinical trial), glucosamine sulfate (e.g., see Rottapharm sponsored clinical trial), diacerein (e.g., see Negma sponsored trial) and doxycycline (see, e.g., NIH sponsored trial). Other suitable DMOADs include calcium pentosan polysulfate (CaPPS) which is a chemically sulfated xylanopyranose, iNOS inhibitors such as L-N6-imminoethyl-L-lysine and SD-6010 (NIH registration NCT00565812), osteogenic protein 1 (OP-1) also known as bone morphogenetic protein 7 (BMP-7), see NIH registration NCT00456157, fibroblast growth factor 18 (FGF-18, NIH registrations NCT00911469, NCT01033994 and NCT01066871), PG-530742 and PG-116800 (NIH registration NCT00041756), Aggrecanase (AGG-523, NIH registrations NCT00427687 and NCT00454298), IL-1Ra (Anakinra, NIH registration NCT00110916), Canakinumab (ACZ885, NIH registration NCT01160822), AMG 108 (NIH registration NCT00110942), Adalimumab (NIH registrations NCT00296894 and NCT00597623), Infliximab (NIH registration NCT01144143), and DLX 105 (NIH registration NCT00819572), A3AR agonist (CF101, NIH registrations NCT01034306 and NCT00428974), PH-797804 (NIH registration NCT01102660), SAR-113945 (NIH registration NCT01113333), Risedronate, SMC021 (NIH registration NCT00704847), Balicatib (NIH registrations NCT00486434 and NCT 00371670), Protelos, Cholecalciferol (NIH registrations NCT01176344 and NCT00599807), strontium ranelate, and the dual COX/5-LOX (5-lipoxygenase) inhibitor known as Licofelone (Merckle GmbH). The discovery of new DMOADs is under active study.

Another advantage of the inclusion of chitosan in an aggregate such as a microsphere is to provide a sustained release of glucosamine to the joint space. Glucosamine enhances the synthesis of hyaluronic acid, a natural component of the joint space. In addition, the glucosamine may exert a stimulatory effect on mucopolysaccharide synthesis that increases the rate of formation of extracellular cartilage matrix. Thus, the chitosan-containing aggregates of the present disclosure may do more than merely carry a pharmaceutical agent, in that they also provide longer term delivery of an embedded or associated agent due to the mucoadhesive properties of the chitosan, and the breakdown of chitosan provides gradual release of glucosamine into the joint space, which may function as disease modifying osteoarthritis drug.

In addition to chitosan breakdown to provide glucosamine, in one aspect the formulations of the present disclosure include glucosamine or glucosamine sulfate. In further addition, the formulations of the present disclosure may include one or more NSAIDs as disclosed herein. The combination of NSAID and glucosamine or glucosamine sulfate may provide synergistic, additive, or subadditive antinociceptive interactions. For example, the combination of NSAID and glucosamine or glucosamine sulfate may provide an anti-arthritic benefit, including a synergistic effect in chronic inflammatory conditions. As another example, the combination of NSAID and glucosamine or glucosamine sulfate may decrease a depression in cartilage proteoglycan synthesis that would be caused by NSAID alone.

In one aspect, the pharmaceutical agent is a biologic, also known as a biologic agent. The biologic may function as a DMOAD, and indeed several of the DMOADs described elsewhere herein are biologics. The biologic may be used to treat osteoarthritis by, for example, inhibiting or modulating the effects of major cytokines, or by providing growth factors with the goal of improve cartilage repair. One such biologic is tanezumab, a humanized monoclonal antibody directed against either (3-NGF or its receptor, high affinity nerve growth factor receptor (TrkA). See, e.g., Chevalier, X., et al., Nature 9:400-410 (2013). Studies suggest that tanezumab should not be co-administered with NSAIDS (see, e.g., Seidel, M. F. and Lane, N. E. Curr. Rheumatol. Rep. 6:583-588 (2012)).

In one aspect, the pharmaceutical agent is a protein molecule called C-type natriuretic peptide (CNP). This protein is naturally-occurring in the body, and may reduce inflammation and aid in the repair of damaged tissue. CNP cannot be used to treat osteoarthritis in patients because it cannot target the damaged area even when the protein is injected into the cartilage tissue. This is because CNP is easily broken down and cannot reach the diseased site. However, by incorporating CNP into formulations of the present disclosure, e.g., within an aggregate as disclosed herein, the CNP may be gradually released and have a beneficial effect in the joint before it is degraded by the body's mechanisms. For instance, it may slow the progression of osteoarthritis and/or begin to repair damaged tissue.

In one aspect, the pharmaceutical agent is platelet-rich plasma (PRP) or autologous conditioned serum. In another aspect, the pharmaceutical agent is a stem cell. These pharmaceutical agents assist in delivering various beneficial materials to the joint, for example, they may provide growth factors, they may provide entities that regulate anti-inflammatory signals, and they may provide entities that modulate angiogenesis. The PRP may provide for shortened blood coagulation time through increased platelet and erythrocyte aggregation. In addition, the PRP may induce an enhanced rate of PDGF-AB and TGFbeta1 release.

Co-administration of chitosan particles with PRP or stem cells to the joint may provide therapeutic benefit due to the wound healing and anti-inflammatory properties of the combination, and the stimulation of hyaluronic acid production. The ability of the chitosan particles to adhere to both platelets and joint tissue enhances residence time for the platelets in joint. Thus, while the platelets may be incorporated into the microsphere, they may also or alternatively adhere to the outside of the microsphere, particularly a microsphere comprising chitosan. Through adhering to chitosan, the platelets will remain longer in the joint space of a knee or other joint. In addition, the breakdown product of chitosan, namely glucosamine, may beneficially modify symptoms and structure of osteoarthritis, which is particularly helpful when chitosan is retained within the joint and degrades gradually, thus effectively providing a slow-release formulation for glucosamine.

The PRP and stem cells provide morphogenic biomaterials that may modulate the natural healing sequence in the joint. For example, in one embodiment chitosan particles as disclosed herein are loaded with platelet lysate and then combined with human adipose-derived stem cells and cultured in vitro. The release of growth factors from the composition may enhance PDGF-AB and TGFbeta1 release. The combination of chitosan and stem cells provide synergism in the regeneration of soft tissues in the joint space.

Thus, in one embodiment the present disclosure provides a composition comprising chitosan particles and cells. Optionally, the composition is an injectable composition. Optionally, the composition comprises stem cells or platelet cells. In a related embodiment, the present disclosure provides a method for cell therapy comprising (a) administering a cell therapy to a subject; and (b) administering chitosan particles to the subject. In optional embodiment, the method may be further characterized by any one or more of the following exemplary parameters: the cell therapy comprises the administration of platelet rich plasma (PRP) to the subject; the cell therapy comprises the administration of stem cells to the subject; the chitosan particles enhance the residence time in the subject of an active agent from the cell therapy; the chitosan particles enhance the efficacy of the cell therapy; the chitosan particles increase the residency time of the cells in the subject; the chitosan particles enhance the anti-inflammatory efficacy of the cell therapy; the chitosan particles increase a release of growth factors to the subject, compared to the release of growth factors in the absence of the administration of the chitosan particles.

As mentioned previously, the formulations of the present invention may include more than one active agent. For example, the formulations may include both PRP and a steroidal or non-steroid agent as disclosed herein. Combining PRP with a NSAID (such as ketorolac or diclofenac) may provide inhibition of cyclo-oxygenase (COX) enzymes, which are a major mediator for inflammation. The combination may also provide desirable analgesic properties. Ketorolac tromethamine inhibits the COX and lipo-oxygenase (LOX) enzymes, and this may prevent the undesirable synthesis of both prostaglandins and leukotrienes. Thus, in one embodiment the formulations of the present disclosure include PRP and an NSAID, in combination with an aggregate as disclosed herein, e.g., chitosan microsparticles.

In one aspect, the pharmaceutical agent is avocado soybean unsaponifiables (often referred to as ASU) which is a natural vegetable extract made from avocado and soybean oils. As a dietary supplement, ASU has been shown in clinical studies to have beneficial effects on osteoarthritis. See, e.g., NIH registration NCT01062737 for the protease inhibitor Piascledine 300.

In one aspect, the pharmaceutical agent is an anthocyanin, or cherry extract or cherry juice extract which contains anthocyanins. Anthocyanins have been discovered to inhibit the COX-1 and COX-2 enzymes, which are the enzymes that are targeted by anti-inflammatory drugs including aspirin, ibuprofen and the arthritis drugs celecoxib and diclofenac. Some anthocyanins contained in cherries may actually be more powerful than aspirin when it comes to fighting inflammation, according to reports in the December 2004 issue of Journal of Biomedicine and Biotechnology.

In one aspect, the pharmaceutical agent discourages the in vivo production and/or activity of a cytokine. Such agents are sometimes referred to as cytokine blockers. For example, TNFα, IL1 and IL6 cytokines have been targeted for arthritis therapy. Antibody agents that are active against TNFα include Infliximab, Adalimumb, Certolizumab pegol, and Golimumab. Etanercept is a fusion protein that is active against TNFα. Anti IL-1 antagonists include the fusion protein Rilonacept and the humanized antibody Canakimumab. The antibody Tocilizumab can be used against IL6. See, e.g., Edwards, C. J., British Medical Bulletin 73-74(1):71-82 (2005); Feldmann, M. J. Clin. Invest. 118(11):3533-3536 (2008); and Singh, R., et al., Curr. Opin. Rheumatol. 17(3):274-279 (2005) for discussion of cytokine blockers. A cytokine blocker may be used a pharmaceutical agent in the compositions of the present disclosure.

In one aspect, the pharmaceutical agent is atorvastatin or a salt thereof, e.g., atorvastatin calcium. A suitable form of atorvastatin is commercially marketed as Lipitor. Atovastatin may be incorporated into a formulation of the present disclosure in order to provide or enhance a chrondroprotective effect.

In one aspect, the pharmaceutical agent is allopurinol. Allopurinol decreases the amount of uric acid in the body when taken regularly. A build up of uric acid in the body can cause the substance to crystallize in the joints that then causes pain attack, such as occurs in gout. With less uric acid in a person's body, crystallization occurs less frequently and less intensively, thus decreasing the harm to joints. Substances that reduce uric acid build up, such as allopurinol, are thus suitable for use in the presently disclosed formulations and methods. Similarly, Febuxostat, classified as a xanthine oxidase inhibitor, as well as Probenecid, both function as a preventive medication by decreasing uric acid levels in the blood, and may be included in the present formulations and methods. Colchicine is also beneficially used in treating and controlling episodes of gout, and is an inhibitor of the motility of polymorphonuclear leukocytes. Thus, colchicine may be included in the formulations and methods of the present disclosure.

In one aspect, the pharmaceutical agent is curcumin, which is a component of the culinary spice turmeric. Turmeric is derived from the rhizomes (underground stems) of the plant Curcuma longa, a member of the ginger family. It is responsible for the yellow color of Indian curry and American mustard. Curcumin, which has powerful antioxidant and anti-inflammatory properties, is the most active constituent of turmeric. Commercial preparations of curcumin contain three major components: diferuloylmethane, demethoxycurcuin and bisdemethoxycurcumin, together referred to as curcuminoids, all of which have anti-inflammatory activity. Curcumin's efficacy at treating active rheumatoid arthritis was studied in a clinical trial conducted in India and reported by Chandran B. and Goel A., Phytother. Res., Mar. 9, 2012. The authors observed that curcumin reduced the swelling and tenderness of joints, and consequently the pain associated with RA.

In one aspect, the pharmaceutical agent is hydroxychloroquine (also known as Plaquenil). Plaquenil is a disease-modifying anti-rheumatic drug (DMARD) that can decrease the pain and swelling due to arthritis. It may prevent joint damage and reduce the risk of long-term disability. See, e.g., Pavelka Jr., K. et al. Annals of the Rheumatic Diseases 48:542-546 (1989). See also the Hera Study Group, The American Journal of Medicine, 98(2):156-168 (February 1995), who reviewed the results of a clinical trial of hydroxychloroquine and concluded that over 36 weeks, hydroxychloroquine had a significant benefit on synovitis, pain, and physical disability of recent-onset rheumatoid arthritis.

In one aspect, the pharmaceutical agent is resveratrol. Nuclear factor kappa B (NF-κB), is a pivotal transcription factor involved in the activation of the TNF-alpha and IL-1beta genes. Activation of NF-κB in synovial cells is a feature seen in arthritis patients. Resveratrol, a polyphenolic, natural phytoalexin found with particularly high levels in grape skin and red wine is a potent and specific inhibitor of TNF-alpha and IL-1beta induced NF-κB activation. See, e.g., Elmali N. et al., Inflammation, 30(1-2):1-6 (April 2007) who reported evidence showing that intra-articular injection of resveratrol may protect cartilage against the development of experimentally induced IA. See also Gao Xuzhu, Ann. Rheum. Dis. 71:129-135 (2012), who report that Resveratrol modulates murine collagen-induced arthritis by inhibiting Th17 and B-cell function.

Another exemplary NF-κB inhibitor which may be used as a pharmaceutical agent in the compositions and methods of the present disclosure is SAR113945, developed by Sanofi and currently undergoing clinical trials (see NCT01463488). This agent is also known as an IKK-β inhibitor.

In one aspect, the pharmaceutical agent is Sulforaphane, which is a compound found in cruciferous vegetables. Brussels sprouts and cabbage are good sources but broccoli is even better. Studies have suggested that sulforaphane possesses anti-cancer and anti-inflammatory properties and now UK researchers have conducted the first major research into the effect of the substance on joint health. See, e.g., Davidson R. K., et al., Arthritis Rheum. 65(12):3130-40 (December 2013), who report that sulforaphane inhibits the expression of key metalloproteinases implicated in osteoarthritis, independently of Nrf2, and blocks inflammation at the level of NF-κB to protect against cartilage destruction in vitro and in vivo.

In one aspect, the pharmaceutical agent is Rebamipide. Rebamipide is an amino acid analogs of 2(1H)-quinolinone found to have gastroprotective action and efficacy to heal experimental gastric ulcers. In addition, Rebamipide has been found to suppress collagen-induced arthritis through reciprocal regulation of Th17/Treg differentiation and heme oxygenase-1 induction. See, e.g., Moon S. J., Arthritis Rheum. (December 2013). These authors found that the inhibitory effects of rebamipide on joint inflammation are associated with recovery from an imbalance between Th17 and Treg cells and activation of an Nrf2/HO-1 antioxidant pathway.

In one aspect, the pharmaceutical agent is strontium ranelate. This drug has been shown to stimulate bone formation and decreases bone resorption. It is licensed and approved in more than 100 countries for treatment of postmenopausal osteoporosis and osteoporosis in men. It is not approved in the United States but is reportedly used off-label for osteoporosis. See, e.g., Reginster J. Y. et al. Osteoporos Int., 23 (Suppl 2):557-S84:OC3 (2012). A phase III, multicenter, clinical trial of men and women 50 years of age or above with a clinical diagnosis of knee osteoarthritis, which was sponsored by Servier Laboratories, found that treatment with strontium ranelate was associated with less progression of cartilage degradation, and that strontium ranelate was well tolerated.

In one aspect, the pharmaceutical agent is capsaicin, also known as 8-methyl-N-vanillyl-6-nonenamide). Capsaicin is the active component of the chili pepper, which is a plant that belongs to the genus Capsicum. In cream form, capsaicin has been described as a safe and effective treatment for arthritis. See, e.g., Deal C. L., et al., Clin. Ther., 13(3):383-95 (May-June 1991).

In one aspect, the pharmaceutical agent is brucine or brucine-N-oxide. Studies by Yin W., J. Ethnopharmacol.; 88(2-3):205-14 (October 2003) showed that these agents were effective in reducing inflammatory agents and signals in vivo. See also studies by Chen, J. et al., International Journal of Nanomedicine, 8:3843-3853 (2013) directed to the intraarticular injection of brucine.

In one aspect, the pharmaceutical agent is a bradykinin inhibitor or antagonist. Bradykinin (BK) is a vasodilator and inflammatory nonapeptide which is generated in the synovium of people with osteoarthritis. It contributes to the initiation and maintenance of inflammation. The intraarticular administration of bradykinin inhibitor, specifically bradykinin B2 receptor antagonist, has been reported to produce a long lasting analgesic effect in patients affected by knee osteoarthritis. The Menarini Group (Italy) has developed a bradykinin antagonist for the treatment of osteoarthritis, called MEM16132 (see, e.g., clinical trial registration NCT01091116) which may be utilized as a pharmaceutical agent in the compositions and methods of the present disclosure.

In one aspect, the pharmaceutical agent is an NSAID known as Diacerein. Diacerein is in a class of substances called anthraquinones. Diacerein has been found to treat osteoarthritis of the knees and hips, and has been approved for sale in Europe. See, e.g., Pelletier J. P., et al., Arthritis Rheum. 43(10):2339-48 (October 2000). Diacerein has a unique mechanism of action compared to other NSAIDS. Diacerein blocks interleukin-1, as opposed to inhibiting the cyclooxygenase (COX) pathway as NSAIDs do. Gastro-intestinal side effects such as diarrhoea and liver disorders have been observed by some people who receive diacerein.

In one aspect, the pharmaceutical agent is lubricin. Lubricin, also known as proteoglycan 4 (PRG4), superficial zone protein (SZP), and megakaryocyte stimulating factor (MSF), is a glycoprotein. It functions as a lubricant in vivo and helps lower the friction in joints. Lubricin also has a chondroprotective feature since it provides synovial fluid with an ability to dissipate strain energy induced by movement. Lubricin is a naturally occurring material, however it also known in recombinant versions. Recombinant lubricin was found to bind to cartilage surfaces, which significantly reduced the cartilage degeneration. See, e.g., Jay G D et al, Arthritis and Rheumatism, 56(11):3662-9, (November 2007) and Zappone B, Jay G D et al, Langmuir, 24(4):1495-1508, (February 2008). Recombinant lubricin is available commercially from, e.g., SBH Sciences (Natick, Mass.).

The primary components of the aggregates (chitosan and/or hyaluronan) are naturally occurring polymers and/or are derived from naturally occurring polymers. These components are biocompatible and may be used in combination with biological therapeutics such also growth factors and cells. For example, compositions of the present disclosure may include stem cells, such as mesenchymal stem cells and adipose derived stem cells (ASCs), which have both proliferative efficiency and multipotency in tissue regeneration. These stem cells may be delivered into a joint space using the compositions and methods of the present disclosure. As another example of a biological therapeutic, the components may be combined with platelet rich plasma (PRP) as discussed in further detail elsewhere herein.

A pharmaceutical agent may be included within or associated with an aggregate that contains one or both of chitosan and hyaluronan. Alternatively, or additionally, a pharmaceutical agent may be in admixture with an aggregate that contains one or both of chitosan and hyaluronan. Alternatively, or additionally, the pharmaceutical agent may be dissolved in the fluid carrier.

Alternatively, or additionally, the pharmaceutical agent may be in an aggregate form, where the aggregate does not also contain chitosan or hyaluronan. In this case, the pharmaceutical agent may be in a microparticulate or other solid particle form, suspended in the carrier. Other aggregate forms of pharmaceutical agent include an encapsulated pharmaceutical agent such as a vesicle or liposomes. When the carrier is an oil-in-water emulsion, the agent may be aggregated in the oil droplets that are suspended in the water.

In one embodiment, the one or more pharmaceutical agents are coated with a polymer and then this polymer-coated agent is incorporated into or onto an aggregate of the present disclosure. An exemplary aggregate is formed from chitosan. The polymer coating may serve to keep the agent within the aggregate, at least in those cases where there is an attractive interaction between the polymer of the coating and the polymer that forms the aggregate. In addition, or alternatively, the polymer coating may serve to sequester the drug and thereby delay its direct contact with the drug receptor of the subject. In other words, the polymer coating will preclude the drug from contacting the cell receptor for as long as the coating is present around the drug. The coating is preferably biodegradable so that it will dissolve or otherwise separate from the drug, and thereby leave isolated drug free to interact with cell receptors. By appropriate selection of the polymer coating, the drug will be maintained within the aggregate for a longer or shorter period of time, and then after the drug has left the aggregate, the coating will, if it is still present around the drug, optionally serve to keep the drug embedded in the membrane and/or gradually degrade to afford a delayed release of the drug to provide direct drug-receptor contact in the subject.

Exemplary polymers that may be used as a coating for the pharmaceutical agent include chitosan, hyaluronic acid, polycaprolactone (PCL), poly(lactide-co-glycolide) (PLGA) and eudragit. Drug may be readily coated by dissolving both the drug and the coating polymer in a suitable solvent or solvent system, and then performing a spray-drying process or lyophilisation process whereby particles of coated drug may be formed. These particles may then be included in a process for forming an aggregate as disclosed herein, using the coated drug in lieu of some or all of the uncoated drug.

In one embodiment, the pharmaceutically active agent is covalently bonded to a polymer that forms the aggregate. The covalent bond may be present directly between a functional group on the active agent and a functional group on the polymer. For example, an active agent with a carboxylic acid group may directly react with a polymer having an amine or hydroxyl group to form an amide or ester linking group, respectively. Alternatively, a spacer molecule may be use which reacts with both the active agent and the polymer. An example of a spacer molecule in the situation of an acidic drug (diclofenac) and an acidic polymer (hyaluronic acid) is disclosed in, e.g., U.S. Pat. No. 7,879,817. Other suitable spacers are known to those skilled in the art, including spacers that link an acidic drug (e.g., diclofenac) to a basic polymer (e.g., chitosan).

While a pharmaceutical agent such as discussed above is present and useful in some embodiments of the compositions and methods of the present disclosure, other embodiments of the invention do not require or include a pharmaceutical agent. For example, an aggregate that includes chitosan, with or without a pharmaceutical agent, that is delivered and retained in the joint space, may provide direct beneficial effects for joint conditions. For instance, chitosan may have a protective effect on articular cartilage of osteoarthritis. Chitosan may also suppress the mRNA expression of MMP-1 and MMP-3 in cartilage during the early stages of OA. Chitosan may also scavenge superoxide and hydroxyl radicals. Other benefits of chitosan-containing formulations are discussed elsewhere herein.

### Carrier and Components Thereof

The present disclosure provides compositions that may be administered to a patient in order to address joint distress or soft muscle distress of the patient. An optional component of each of these embodiments is either a carrier or the components from which the carrier may be formed.

In a one embodiment, the carrier is a liquid. A liquid carrier is particularly preferred when the composition will be administered to the patient by injection or like means, e.g., by catheter. The liquid component of the carrier may be water, and a carrier that includes water may be referred to herein as an aqueous carrier.

The aqueous carrier may contain dissolved salts, where suitable salts include sodium phosphate, potassium chloride, sodium chloride, potassium phosphate and the like. These salts are included in the carrier in order to adjust the osmolarity and ion concentration of the composition to those of the subject receiving the composition. In one aspect the carrier is PBS buffer, which is an aqueous solution of containing one or more dissolved salts such as sodium phosphate to provide an isotonic solution.

The aqueous carrier may additionally, or alternatively, contain dissolved hyaluronan, referred to herein as dissolved nAH. nAH is a natural component of synovial fluid, and is beneficial in providing viscoelasticity to the joint space. The inclusion of nAH in a composition of the present disclosure thus helps overcome the loss of natural hyaluronan from the patient's joint space.

The concentration of nAH present in the carrier can vary, but in an exemplary embodiment nAH is provided at a pharmaceutically effective amount. In other exemplary embodiments, the nAH is present in the liquid carrier at a concentration of 1-5 mg/ml, at least about 5 mg/ml, 1-7 mg/ml, at least about 7 mg/ml, 1-10 mg/ml, at least about 10 mg/ml, 1-15 mg/ml, at least about 15 mg/ml, 1-20 mg/ml, at least about 20 mg/ml.

Another optional component of the aqueous carrier crosslinked hyaluronan. For example, the carrier may be SYNVISC® (hylan G-F 20) or SYNVISC-ONE®, the former being an elastoviscous high molecular weight fluid containing hylan A and hylan B polymers produced from chicken combs. The hyaluronan in Hylan G-F 20 is chemically crosslinked. These materials are sold by Sanofi-Aventis U.S. LLC (Cambridge, Mass.).

The aqueous carrier may additionally, or alternatively, contain dissolved chitosan, referred to herein as dissolved nAC. The inclusion of nAC in a composition of the present disclosure may assist in increasing the viscosity of the composition. The in vivo degradation product(s) of chiotosan may also contribute to the therapeutic benefits of the present compositions and methods. As mentioned previously, chitosan has been shown to have an analgesic effect on inflammatory pain (see, e.g., Okamoto Y., et al., Carbohydrate Polymers 49:249-252 (2002)), perhaps by absorbing excess protons. Thus, nAC may provide the benefit of reducing pain associated with joint distress, perhaps due to the fact that chitosan breaks down, in vivo, to glucosamine, which is a DMOAD discussed later herein.

The concentration of nAC present in the carrier can vary, but in an exemplary embodiment nAC is provided at a pharmaceutically effective amount. In other exemplary embodiments, the nAC is present in the liquid carrier at a concentration of 1-5 mg/ml, at least about 5 mg/ml, 1-7 mg/ml, at least about 7 mg/ml, 1-10 mg/ml, at least about 10 mg/ml, 1-15 mg/ml, at least about 15 mg/ml, 1-20 mg/ml, at least about 20 mg/ml.

The formulations may contain polyhydric alcohol e.g., mannitol, sorbitol or trehalose. These polyhydric compounds may act as an antioxidant to prevent hyaluronic acid from depolymerizing. The formulations may also, or alternatively contain carnosine, which is an anti-oxidation compound that may reduce degradation of HA.

### Optional Components

When it is desired to crosslink the chitosan by means other than by forming ionic complexes with hyaluronan, crosslinking may be achieved by reacting the chitosan with polyfunctional materials such as tripolyphosphate (TPP), glutaraldehyde or genipin. Genipin, also known as cyclopenta(c) pyran-4-carboxylic acid, 1,4a-alpha,5,7a-alpha-tetrahydro-1-hydroxy-7-(hydroxymethyl)-, methyl ester, is seeing increased use as a crosslinking agent since it is a natural product. Genipin is a hydrolytic product of geniposide, which is found in the fruit of Gardenia jasminoides Ellis. The mechanism of genipin facilitated crosslinking with chitosan is unclear, but likely involves reaction with the primary amine groups of chitosan. Genipin is commercially available from, e.g., Challenge Bioproducts Co., Ltd., Taiwan.

The crosslinking of chitosan not only impacts the degradation rate of chitosan aggregates, but it also impacts the degree of swelling that a chitosan aggregate undergoes when exposed to moisture. In general, greater crosslinking will mitigate the amount of swelling which chitosan aggregates undergo when placed into aqueous media such as PBS. For purposes of the present disclosure, excess swelling is undesirable since larger aggregates are more difficult to deliver through a fixed size of syringe needle or cannula. Also, in general, dried aggregates of the present disclosure may swell when exposed to water (be rehydrated), thus increasing their average particle size.

While a crosslinking agent may be utilized in some embodiments of the compositions and methods of the present disclosure, the crosslinking agent is an optional feature. In some embodiments, TPP is not included in the compositions and methods. In another embodiment no additional crosslinker is included in the compositions and methods, i.e., crosslinking occurs only by interaction between the chitosan and the hyaluronan.

However, when a crosslinking agent is used, the present disclosure provides an embodiment which is a composition comprising particles, where the particles comprise crosslinked chitosan having amino (—NH₂) groups. The crosslinked chitosan may be the reaction product of chitosan and a crosslinking agent, preferably a crosslinking agent that covalently reacts with amino groups present on the chitosan, such as glutaraldehyde. In order to assure the presence of amino groups on the crosslinked chitosan, the chitosan may be the deacetylation product from chitin, where the chitosan has a relatively high degree of deacetylation (DDA) of greater than 75% and less than 95%, where this high DDA provides for a large number of amino groups on the chitosan. The large number of amino groups on the starting chitosan allows that some of those amino groups may react with the crosslinking agent, such as glutaraldehye, to form crosslinks, while leaving other of those amino groups as unreacted —NH₂ groups which may protonate to form ammonium (—NH₃⁺) groups when the composition is administered into a subject. It may be mentioned that not all of the chitosan polymers present in the particle are necessarily crosslinked. The protonated form of crosslinked chitosan is advantageous since the ammonium groups interact with biological tissue to form an adhesive interaction between the particles and the tissue. The particles of any of the compositions as disclosed herein may be generally spherical and may be characterized by a diameter. Even though a particle may not be perfectly spherical, it may be considered generally spherical and be characterized by a diameter, where the diameter of a spherical or non-spherical particle refers to the longest straight line that may be drawn that intersects the outer edges of the particle. In various embodiments a composition comprises particles with diameters within the range of at least 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or at least 40 microns, and up to 120, or 119, or 118, or 117, or 116, or 115, or 114, or 113, or 112, or 111, or 110, or 109, or 108, or 107, or 106, or 105, or 104, or 103, or 102, or 101, or 100, or 99, or 98, or 97, or 96, or 95, or 94, or 93, or 92, or 91, or up to 90 microns. For example, the particles may have diameters of greater than 25 microns and less than 100 microns, or greater than 30 microns and less than 100 micron. Unless otherwise stated, it should be understood that a composition of the present disclosure may have some particles with diameters below a stated minimum value, e.g., less than 25 microns, and that such a composition is still a composition of the present disclosure so long as at least some of the particles in the composition have diameters within the stated range. Likewise for compositions which have some particles greater than a stated upper range value. When the particles become too large, e.g., above 110 microns, they are difficult to administer via a syringe having a needle that is of a comfortable gauge to be tolerated by an average subject in need of treatment. In one embodiment, the composition further comprises a liquid medium, and the composition is injectable through a needle of 18-27 gauge. Particles having diameters within a suitable range, e.g., 25-100 microns, may be prepared by, e.g., spinning disk technology. The particles of any of the embodiments of the present disclosure may be prepared from chitosan having an intrinsic viscosity of at least 1 mPas and less than 1,000 mPas, and ranges selected from values contained therein. For particles prepared by spinning disk technology, a preferred chitosan has a viscosity in the range of about 50 to about 300 mPas. In general, the starting chitosan may have an intrinsic viscosity of greater than 1 mPas, or greater than 10, or 20, or 30, or 40, or 50, or 60, or 70, or 80, or 90, or 100 or 110, or 120, or 130 or 140 or 150 mPas, and less than 1,000 mPas, or less than 900, or 800, or 700, or 600, or 500, or 400, or 300, or 290, or 280, or 270, or 260, or 250, or 240, or 230, or 220, or 210 or 200 mPas. The particles may incorporate an active pharmaceutical ingredient (API) as disclosed herein, where the API is optionally an NSAID, and is optionally diclofenac. The API, which may be diclofenac, may optionally be present within the particle in a crystalline form, where in optional embodiments substantially all of the API is in crystalline form, or most of the API is in crystalline form, or at least some of the API is in crystalline form, or a minor proportion of the API is in crystalline form and a majority of the API is in amorphous form.

Thus, in one embodiment the present disclosure provides a composition comprising particles, the particles comprising crosslinked chitosan having amino (—NH₂) groups, and an active pharmaceutical ingredient (API) incorporated into the particles, wherein the API is optionally diclofenac and at least some of the diclofenac is in crystalline form, the particles having a diameter of, e.g., greater than 10, or 15, or a 20, or 25, or 30 microns, and up to, e.g., 120, or 110, or 105, or 100, or 95 microns, wherein the crosslinked chitosan is the reaction product of a crosslinking agent and chitosan, where the crosslinking agent may form covalent or non-covalent crosslinks although optionally the crosslinking agent forms covalent crosslinks such as are formed when glutaraldehyde is the crosslinking agent, and the chitosan has a degree of deacetylation (DDA) of, e.g., greater than 75%, or greater than 80%, or greater than 85% and less than 95%, and has an intrinsic viscosity of, e.g., greater than 50 mPas or greater than 100 mPas and less than 300 mPas or less than 250 mPas. The composition may further comprise a liquid medium such as water or aqueous buffer or other aqueous carrier, to provide a liquid composition that is injectable through a needle of 18-27 gauge. In optional embodiments, the only polymers present in the particles are crosslinked chitosan, or crosslinked chitosan and non-crosslinked chitosan. The particles of the present disclosure may be referred to as chitosan particles, which unless the context indicates otherwise, refers to particles that are prepared from chitosan and may or may not have crosslinking between the chitosan chains.

Thus, in one embodiment the present disclosure provides a composition comprising particles, the particles comprising crosslinked chitosan having amino (—NH₂) groups, and an active pharmaceutical ingredient (API) incorporated into the particles, wherein the API is diclofenac and at least some of the diclofenac is in crystalline form, the particles having a diameter of greater than, for example, 25 microns and less than, for example, 100 microns, wherein the crosslinked chitosan is the reaction product of glutaraldehyde and a chitosan having a degree of deacetylation (DDA) of greater than, for example, 75% and less than, for example, 95%, and having an intrinsic viscosity of greater than, for example, 50 mPas and less than, for example, 300 mPas. As mentioned elsewhere herein, the particles of any of the embodiments of the present disclosure may be prepared from chitosan having an intrinsic viscosity of at least 1 mPas and less than 1,000 mPas, and ranges selected from values contained therein. For particles prepared by spinning disk technology, a preferred chitosan has a viscosity in the range of about 50 to about 300 mPas. In general, the starting chitosan may have an intrinsic viscosity of greater than 1 mPas, or greater than 10, or 20, or 30, or 40, or 50, or 60, or 70, or 80, or 90, or 100 or 110, or 120, or 130 or 140 or 150 mPas, and less than 1,000 mPas, or less than 900, or 800, or 700, or 600, or 500, or 400, or 300, or 290, or 280, or 270, or 260, or 250, or 240, or 230, or 220, or 210 or 200 mPas. The composition may further comprise a liquid medium such as water or aqueous buffer or other aqueous carrier, to provide a liquid composition that is injectable through a needle of 18-27 gauge. In optional embodiments, the only polymer present in the particles is crosslinked chitosan, or alternatively a mixture of crosslinked chitosan and non-crosslinked chitosan. However, in other embodiments, a polymer other than chitosan or crosslinked chitosan may be present in the particle.

As discussed elsewhere herein, this composition may be used in a method of delivering a particle into a synovial fluid and a synovium of a subject to provide a depot for sustained release of an active pharmaceutical ingredient (API) from the particle, the method comprising: (a) providing a liquid composition as described above, comprising particles, the particles comprising crosslinked chitosan having amino (—NH2) groups, and an active pharmaceutical ingredient (API) incorporated into the particles, the particles having a diameter of, e.g., greater than 25 microns and less than 100 microns (other options are a composition comprising particles with diameters within the range of at least 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or at least 40 microns, and up to 120, or 119, or 118, or 117, or 116, or 115, or 114, or 113, or 112, or 111, or 110, or 109, or 108, or 107, or 106, or 105, or 104, or 103, or 102, or 101, or 100, or 99, or 98, or 97, or 96, or 95, or 94, or 93, or 92, or 91, or up to 90 microns), wherein the crosslinked chitosan is the reaction product of a crosslinking agent and a chitosan having a degree of deacetylation (DDA) of, e.g., greater than 75% and less than 95%; (b) providing a syringe containing the liquid composition, the syringe having a needle in the range of 18-27 gauge; and (c) intra-articularly injecting the liquid composition through the needle and into a subject in need thereof.

The present disclosure also provides, in one embodiment, an injectable composition comprising an aqueous carrier and cross-linked chitosan particles dispersed in the aqueous carrier, wherein at least one of the following is true: (a) the majority of the particles have a diameter of, for example, between 25 and 100 microns (other options are a composition comprising particles with diameters within the range of at least 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or at least 40 microns, and up to 120, or 119, or 118, or 117, or 116, or 115, or 114, or 113, or 112, or 111, or 110, or 109, or 108, or 107, or 106, or 105, or 104, or 103, or 102, or 101, or 100, or 99, or 98, or 97, or 96, or 95, or 94, or 93, or 92, or 91, or up to 90 microns); (b) the chitosan has a degree of deacetylation (DDA) of, for example, 75-95%; and/or (c) the chitosan has an intrinsic viscosity of, for example, 50-300 mPas; where the composition further comprises an active pharmaceutical ingredient (API). In additional embodiments, the present disclosure provides an injectable composition characterized by any one or more of the following exemplary features: the API is an NSAID; the API is diclofenac; the API is diclofenac, and at least some of the diclofenac is in crystalline form; the API is diclofenac, and substantially all of the diclofenac is in crystalline form; the API is diclofenac, and the diclofenac is released from the particles in a therapeutic non-inflammatory concentration; the composition has a low, i.e., non-inflammatory, endotoxin concentration.

In another embodiment, the present disclosure provides a composition that achieves both immediate and extended release of an active pharmaceutical ingredient (API) upon administration to a subject, the composition comprising chitosan particles and API, the release occurring through at least one of diffusion of API from the particles and breakdown of the particles to release entrapped API. In additional embodiments, the present disclosure provides a composition that achieves both immediate and extended release of an API which is characterized by any one or more of the following exemplary features: the chitosan particles comprise crosslinked chitosan; the API is an NSAID; the API is diclofenac which optionally may be in whole or in part in crystalline form; the immediate release occurs over a period of 12 hours from the administration, and releases from 5 to 70% of API from the particles; the extended release occurs over a period of 0.5 to 7 days from administration, and releases 5 to 70% of the API from the particles; the particles are taken up by the synovium after administration to the subject. For example, administration of crosslinked chitosan particles incorporating diclofenac crystals to the knees of sheep was found to provide a synovial fluid concentration of diclofenac of 0.69 mg/mL after 7 days following intra-articular administration, and 0.13 mg/mL after 14 days following intra-articular administration. In contrast, direct intra-articular injection of the equivalent amount of diclofenac (without being incorporated into a chitosan particle) yielded a concentration of about 0.05 mg/mL of diclofenac in the synovial fluid after 7 days following the injection, and after 14 days following the injection there was no detectable diclofenac in the synovial fluid. Accordingly, the incorporation of API into a crosslinked chitosan particle as disclosed herein may provide an extended release of API into the synovial fluid.

In a further embodiment, the present disclosure provides a composition comprising chitosan and diclofenac, wherein at least some of the diclofenac is in a crystalline form. In additional embodiments, the present disclosure provides a composition comprising chitosan and diclofenac, wherein at least some of the diclofenac is in a crystalline form, which is characterized by any one or more of the following exemplary features: the diclofenac is in both crystalline and amorphous form; a major proportion of the diclofenac is in crystalline form and a minor proportion of the diclofenac is in amorphous form; substantially all of the diclofenac is in crystalline form; a major proportion of the diclofenac is in amorphous form and a minor proportion of the diclofenac is in a crystalline form; the chitosan is in particle form, and the diclofenac is incorporated into the particles; the composition further comprises aqueous carrier; the diclofenac is present at a range of 1-25% of the total weight of chitosan, crosslinked chitosan and diclofenac present in the composition.

In another embodiment, the present disclosure provides a composition comprising chitosan and an active pharmaceutical agent (API), wherein at least some of the API is in an amorphous form. In additional embodiments, the present disclosure provides a composition comprising chitosan and API, wherein at least some of the API is in amorphous form, which is characterized by any one or more of the following exemplary features: the API is in both crystalline and amorphous form; all of the API is in amorphous form; a major proportion of the API is in crystalline form and a minor proportion of the API is in amorphous form; substantially all of the API is in amorphous form; a major proportion of the API is in amorphous form and a minor proportion of the API is in a crystalline form; the chitosan is in particle form, and the API is incorporated into the particles; the composition further comprises aqueous carrier; the API is present at a range of 1-25% of the total weight of chitosan, crosslinked chitosan and API.

In another embodiment, the present disclosure provides a composition comprising crosslinked chitosan and an active pharmaceutical agent (API). Optionally, the API is an NSAID, where the NSAID may optionally be diclofenac. The composition may contain particles, where optionally at least some of the particles have a diameter within the range of, for example, 25 to 100 microns. Preferably, the composition contains little or no unreacted crosslinking reagent.

An aqueous carrier includes water and may additionally include one or more optional components such as inorganic or organic salt, sugar such as dextrose, preservative, and buffering agent.

The aqueous carrier may optionally contain blood plasma, such as platelet rich plasma (PRP), and autologous or allogeneic conditioned serum. The blood plasma contains growth factors and/or inflammatory mediators that may contribute to the prevention of joint degeneration progression and enhance the repair process, particularly in joints that have osteoarthritis. In one embodiment, the PRP is autologous blood with a concentration of platelets above baseline, e.g., about 400% of the peripheral blood platelets count and/or having 1 million platelets or more per millimeter. Methods to prepare PRP are described in, e.g., Dohan et al. Trends in Biotechnol. 27(3):158-167, 2009. The intra-articular injection of PRP has been reported to be useful in the early stages to modulate inflammatory processes. See, e.g., Frizziero, A., Sport Sci. Health, 8:15-22 (2012).

### Aggregate and Carrier Preparation and Properties

Aggregates suitable for the compositions and methods of the present disclosure may be prepared by any of a number of processes. Exemplary processes include spray drying and emulsion techniques, each of which is described below.

The aggregates may be prepared by a spray drying process. Spray-drying may be used to prepare aggregates from, for example, solutions, suspensions, emulsions and molten forms of a composition. Such processes are known in the art, see, e.g., Ka\check{s}par, 0., et al., Powder Technology 240:31-40 (2013) and references cited therein. Ka\check{s}par et al disclose methodology for preparing chitosan-TPP crosslinked aggregates using spray drying techniques. For example, separate solutions of chitosan and TPP may be prepared and then separately fed into the outer and inner sections of a 3-fluid nozzle, respectively. Varying the ratio of their flow rates or concentrations can be used to effect average aggregate size and morphology. A higher TPP/chitosan ratio generally leads to a high mean aggregate size, however the aggregate size also depends on the concentration of the spray-dried colloidal solution. A mean aggregate size of 2-4 μm is generally available by this technique. To create larger sized aggregates, a 2-fluid nozzle system using chitosan solutions of about 0.5% (w/v) may be employed, to provide an aggregate size up to about 10 μm with a span (D₉₀-D₁₀) of about 3-10 μm.

As another example, chitosan aggregates may be prepared by dissolving chitosan (MW is 600 kDa, DDA is 96%) at 0.75% w/v concentration in 1% acetic acid in water (v/v). Other suitable concentrations of chitosan include 1%, 2% and 3% (w/v). Glutaraldehyde (1% v/v) is added to a ratio of 1:20 (glutaraldehyde:chitosan=1:20 v/v) with stirring for 1 hour. The aggregates are prepared by spray-drying using Q-flow at 40 m³/hr, exit temperature at 120° C. and pump speed of 15 mL-min⁻¹. Inclusion of pharmaceutical agent in the aggregate may be accomplished by dissolving or suspending small particulates of the pharmaceutical agent in the aqueous chitosan solution. The aggregates have a size distribution ranging from 0.5 to 4.5 μm, with an average size of about 2.5 μm. For further information, Desai, K. G. H., et al., Drug Development Research 64:114-128 (2005), provides a good overview of spray drying techniques for forming chitosan aggregates.

Spray-drying may also be accomplished using a spinning disk apparatus rather than a nozzle to form the tiny droplets which, after solvent removal from those tiny droplets, provide the aggregates, e.g., particles or microparticles, of the present disclosure. The spinning disk apparatus may be that described in U.S. Pat. No. 7,758,778, or one of the several spinning disk apparatuses mentioned therein. In order to provide aggregates that include a crosslinking agent, e.g., chitosan crosslinked with TPP, the TPP and chitosan may be combined at any of various stages of the aggregation-formation process. For example, the chitosan and TPP may be combined in a suitable solvent (such as acidic water, e.g., a mixture of water and acetic acid) along with the active agent (such as diclofenac) to provide a suspension or solution that may be deposited onto the spinning disk, or into a reservoir on a spinning disk. Alternatively, a solution of TPP may be added to the reservoir on a spinning disk, and a solution or suspension of chitosan is separately added to the reservoir on the spinning disk, where the active agent may be a component of either the TPP or chitosan solution/suspension. Upon being combined within the reservoir, the crosslinking agent and the chitosan begin to from aggregates which are present in the tiny droplets which are expelled from the spinning disk.

Thus, in one embodiment the present disclosure provide particles prepared by adding reactants to a spinning disk process, the reactants comprising chitosan, crosslinking agent for chitosan, and active pharmaceutical ingredient (API), where the resulting particles comprise crosslinked chitosan and API. Any one of more of the following exemplary optional features may further characterize the particles prepared by a spinning disk process: the crosslinking agent is glutaraldehyde; the crosslinking agent is TPP; the API is an NSAID; the API is diclofenac, at least some of which is optionally present in the resulting particles in a crystalline form. The resulting particles may have an essentially spherical form and a diameter in a range of, for example, about 15, or 20 or 25 or 30 to about 120 or 115, or 110, or 105 or 100 microns.

In a related embodiment, the present disclosure provides a method of forming particles from chitosan, crosslinking agent for chitosan, and active pharmaceutical ingredient (API). The method comprises: (a) combining chitosan and API in a suitable solvent such as a mixture of water and methanol to provide a uniform solution or suspension; (b) adding crosslinking agent, such as glutaraldehyde with stirring to the uniform solution or suspension of (a); (c) adding the mixture from (b) to a spinning disk apparatus in a gradual, e.g., dropwise, manner to allow particle formation.

When a spray drying apparatus is used, in one embodiment the aggregates that come from the nozzle or spinning disk or the like, after they are optionally dried in a drying chamber (typically with the assistance of a dry inert gas) may be delivered into a liquid bath. The bath may allow any residual solvent contained within the aggregate to diffuse into the bath and thus away from the aggregate. In addition, or alternatively, the bath may contain one or more components that react with and/or can be incorporated into the aggregate. For instance, the crosslinking agent (such as TPP) may be present in the bath and undergo a reaction with the non-crosslinked polymer (such as chitosan) in order to provide a crosslinked aggregate. As another example, pharmaceutical agent may be present in the bath, where that agent may, for example, diffuse into the aggregate, or complex with the aggregate so as to be the sole agent in the composition, or so as to supplement agent that was already incorporated into the aggregate during aggregate formation, or so as to add a new pharmaceutical agent that was not already incorporated into the aggregate during aggregate formation, optionally thereby providing a layered distribution of pharmaceutical agent(s) in the aggregate. As yet another example, in the event that acid or base is present in the initial formulation that is delivered to the spray drying apparatus, the bath may contain base or acid as need to neutralize that acid or base present in the initial formulation.

The aggregates of the present invention may include a core-shell configuration. For example, a core containing drug diffused throughout a polymer matrix, and a shell that surrounds the core where the shell is typically a polymer matrix different from the polymer that is present in the core. Such core-shell aggregate, may be prepared as described in U.S. Pat. No. 7,758,778, where they are described as microcapsules. In one embodiment, the core and shell polymers are selected from hyaluronic acid and chitosan. In another embodiment, chitosan is present in the core but not the shell. In yet another embodiment, chitosan is present in the shell but not the core. In still another embodiment, chitosan is present in both the core and the shell.

The aggregates may be prepared by an emulsion process, where such processes are known in the art. See, e.g., Kotsaeng, H., et al., Particulate Science and Technology 28:369-378 (2010), and references cited therein. Kotsaeng et al. describe a variation on the emulsion process called a W/O emulsification-diffusion method. According to this process, an aqueous chitosan solution provides the water phase and ethyl acetate provides the oil phase for forming an oil-in-water emulsion. Chitosan solutions are prepared by dissolving chitosan flakes (85-90% DDA, average molecular weight of 100 or 740 KDa) into a 4% (v/v) acetic acid solution. This solution was added drop-wise to ethyl acetate, and then the container was covered to prevent ethyl acetate evaporation while stirring continued for one hour. A stirring rate of about 600 rpm was found to be about optimum. The aggregates were collected by centrifugation and dried in a vacuum oven for 24 hours at room temperature. Cross-linking of the aggregates may be achieved by post-treatment with glutaraldehyde. In this post-treatment process, the chitosan aggregates are immersed in a 2.5% (v/v) glutaraldehyde solution with moderate shaking for 24 hours at room temperature. Thereafter, the particles are collected by centrifugation and washed with distilled water prior to drying in a vacuum oven at 40° C. The size of the aggregates ranged from less than 150 μm to greater than 300 μm. Aggregate morphology may be effected by varying the concentration of chitosan in the starting solution. Aggregates prepared by the process of Kotsaeng may be spongy, i.e., porous. In one aspect, an aggregate of the present disclosure is porous.

Aggregates of chitosan, optionally incorporating pharmaceutical agent, may be prepared by an emulsion / chemical crosslinking technique as described in Ge Y. B., et al., Int. J. Pharm. 338:142-151 (2007), and Ge Y. B., et al. Yakugaku Zasshi 131(12):1807-1812 (2011). According to this technique, a solution of chitosan (5% w/v in water, DDA greater than 80%) with suspended pharmaceutical agent (in e.g., micronized form) having a polymer:drug weight ratio of about 1:1.2. This aqueous phase is dropped into an oil phase consisting of liquid paraffin containing 2% Span 80 (w/v) to form a water in oil emulsion by stirring at 300 rpm for 15 minutes. Then 25% glutaraldehyde was added and stirred for 3 hours at room temperature. The crosslinked microspheres were separated and washed by vacuum-induced filtration, and then dried in a vacuum desiccator at room temperature. The resulting microspheres have a mean particle diameter of about 73 μm. A similar emulsion-based technique for making drug loaded chitosan aggregates is described in Mothilal, M. Int. J. Pharma Sciences and Res. 3(2):305-315 (2012).

Other processes which may be employed to prepare isolated aggregates suitable for the present compositions and methods include precipitation and granulation. For example, a 2.5 mg/mL chitosan solution may be prepared in a mixture of 2% (v/v) acetic acid and 1% (w/v) Tween 80 surfactant. Then 10% (w/v) aqueous sodium sulfate solution is added dropwise to the chitosan solution under continuous sonication. After adding the sodium sulfate, stirring and sonication is continued for 20 minutes. Aggregates formed by this method are separated by centrifugation at 4,000 rpm and may be dried to provide a dry composition of the present disclosure. The dried aggregates may subsequently be re-suspended in water to provide a liquid composition of the present disclosure.

In addition, the following articles provide information about the preparation of aggregates. Zambito, Y. Advances in Biomaterials Science and Biomedical Applications, Chapter 9, pages 243-263 (2013); Rafeeq, M. et al. Research J. Pharmaceutical, Biological and Chemical Sciences 1(4):383-390 (October-December 2010); and specifically in regard to ionotropic gelation, see, e.g., Koukaras, E. N., et al. Molecular Pharmaceutics 9:2856-2862 (2012).

As mentioned previously, a crosslinking agent may be used in combination with the chitosan in order to stabilize the chitosan aggregate. When TPP is selected as the crosslinking agent, a stabilized chitosan aggregate may be prepared as follows. A solution of chitosan (2.5 mg/mL chitosan in solution) in 2% (v/v) acetic acid/water is prepared, optionally with 1% (w/v) surfactant, e.g., TWEEN 80. A solution of TPP in water is prepared at a concentration of 3.0 mg/mL and added dropwise to the chitosan solution under magnetic stirring with total volume at about 4.0 mL. Final concentration of chitosan in solution is in the range of 1-3 mg/mL and final TPP concentration is in the range of 0.5-1 mg/mL.

Aggregates may be characterized by particle size, where particle size may be measured by, for example, suspending aggregate in hexane or water and using a Malvern Mastersizer Model MS/S with 300RF mm lens, which measure particle size by laser light scattering. This instrument is available from Malvern Instruments (Malvern, U.K.). Particle size may also be measured by optical microscopy, by transferring a suspension of the aggregates to a microscope slide and then evaporating the solvent. In one aspect, an aggregate of the present disclosure has an average particle size in the micron range, i.e., a mode cross-sectional distance of from 1 to 1000 microns, or 1 to 500 microns, or from 5 to 500 microns, or from 5 to 100 microns, or from 5 to 50 microns. In another aspect, the aggregates have a mode particle size in the nanometer range, i.e., an average cross-sectional distance of from 1 to 1000 nanometers. In another aspect, the average particle size spans the nanometer and micrometers ranges, e.g., having a mode cross-sectional distance of from 100 to 100,000 nanometers (0.1 to 100 microns) or from 100 to 50,000 nanometers (0.1 to 50 microns) or from 500 to 100,000 nanometers (0.5 to 100 microns) or from 500 to 50,000 nanometers (0.5 to 50 microns). The particle size may also be described by a distribution. In one aspect, the particle size distribution is narrow, e.g., about 90% of the particles have a particle size that is within 20% of the mode particle size.

The particle size of the aggregates may change depending on whether they are exposed to fluid. A lyophilized aggregate may have a smaller particle size than a re-suspended aggregate. Unless otherwise clear from the context, the particle sizes mentioned herein refer to suspended particles as analyzed by optical microscopy.

As mentioned elsewhere herein, in various aspects the aggregates have a minimum particle size of 1, or 5, or 10, or 15, or 20, or 25, or 30, or 35, or 40, or 45, or 50 microns. In addition, or alternatively, the aggregates may be characterized in terms of a maximum particle size of 100, or 95, or 90, or 85, or 80, or 75, or 70, or 65, or 60, or 55, or 50, or 45, or 40, or 35, or 30 microns. The aggregates may optionally be characterized in terms of having particle sizes that fall within a range, where upper and lower limits of the range of may be selected from the preceding values. For instance, the aggregates may have a particle size within the range of 10-80 microns, or within the range of 20-70 microns, or within the range of 20-40 microns, or within the range of 20-30 microns, as four examples.

Noteworthy is that when the aggregates are characterized in terms of particle size range, specifying a lower and upper limit to the range, this is not to say that all of the aggregates in a composition of the present disclosure have a particle size that necessarily falls within the scope of the stated range. Rather, the specification that the aggregates have a particle size in the range of x-y (x is a lower limit to a range, y is an upper limit to a range) means that at least some of the aggregates have a particle size within the stated range, and that those specific aggregates may or may not be associated (e.g., in the same solution as) with aggregates that have particle sizes outside the scope of the range. In various embodiments, a composition of a plurality of aggregates may be characterized in terms of the numerical fraction of the aggregates that have particle sizes that fall within a stated range, or that fall below a stated maximum size, or that have sizes greater than a stated minimum size. For example, a composition of aggregates may be characterized as having at least 95%, or at least 90%, or at least 85%, or at least 80%, or at least 75%, or at least 70%, or at least 65%, or at least 60%, or at least 55%, or at least 50% of the number of aggregates in the composition as having particle sizes that fall within the stated range x-y of particle size.

The aggregates may also be characterized in terms of the identity, quantity and/or morphology of a pharmaceutical agent that is associated with the aggregate. In general, pharmaceutical agents may exist in an amorphous form within and/or on the surface of the aggregates, or they may exist in a crystalline form, at least for those agents that can crystallize. In one embodiment, the aggregates of the present disclosure associate with an amorphous form of a pharmaceutical agent. In another embodiment, the aggregates of the present disclosure associate with a crystalline form of a pharmaceutical agent. In yet another embodiment, the aggregates associate with both crystalline and amorphous forms of a pharmaceutical agent, where the agent may be, for example, diclofenac. The term “associate” is used to include agent that is either embedded within the aggregates, or agent that is complexed with the surface of the aggregate.

The form in which the pharmaceutical agent associates with the polymer of the aggregate may impact the kinetics of the release of the agent from the aggregate. For example, amorphous active agent that is dispersed within the aggregate may release by a slow diffusion process. In a slow diffusion process, the dispersed agent equilibrates with the surrounding fluid, thereby diffusing out from the aggregate and into the adjacent tissue or fluid of the host. With the aggregates of the present disclosure, a slow diffusion process may release pharmaceutical agent over a period of days or weeks. As another example, when the agent is in a crystalline form, and is embedded within the aggregate, it may not become free of the aggregate until the polymer of the aggregate degrades and provides a physical opening for the crystal to exit the aggregate. With the aggregates of the present disclosure, the release of crystalline pharmaceutical agent may take place over a period of days or weeks or months, where such a release may be referred to as an extended release, and the aggregate may be referred to as a depot. The aggregates of the present disclosure may contain both amorphous and crystalline pharmaceutical agent, in which case the release of pharmaceutical agent may initially be a slow diffusion process, followed by an extended release process, where there may an overlap between the two release processes, such that both slow diffusion and extended release occurs simultaneously.

Whether the pharmaceutical agent is in a crystalline or amorphous form therefore may impact the release profile of the agent, when that agent is contained within the aggregate. When the agent is associated with the surface of the aggregate, the form of the agent has a smaller effect on release profile. In other words, when the agent is associated with the surface, or near surface, of the aggregate, it will readily leave the aggregate by diffusion (in the case of amorphous agent) or dissolving and then diffusing (in the case of crystals). The release of the agent from the surface or near surface of the aggregate occurs rapidly and provides for delivery of an initial bolus or burst of agent to the subject.

In one embodiment, the pharmaceutical agent is an NSAID such as diclofenac. In one embodiment, the NSAID, e.g., diclofenac, is present in the aggregate in a crystalline form. In another embodiment, the NSAID, e.g., diclofenac, is present in the aggregate in both a crystalline form and an amorphous form. The agent may crystallize in the aggregate during aggregate formation, as illustrated by an Example provided herein.

In one embodiment, the majority of the aggregates in a composition of the present disclosure have a particle size in the range of 20-50, or 30-40 microns. Optionally, less than 5% of the aggregates have a particle size of more than 60 microns. It is observed that aggregates in the size range of 20-50 or 30-40 microns are particularly advantageous in that they readily embed in the membrane that lies at the edges of a joint space, e.g., the synovial membrane of a knee joint. This particle size, in combination with a net positive charge for the particles, is particularly suited for embedding in the membrane. Upon being embedded in the membrane, the aggregates release the pharmaceutical agent, either by providing slow diffusion of amorphous agent from the aggregate, or by functioning as a depot for release of crystalline form of the active agent. When the aggregate size is smaller, typically under 20 microns or under 10 microns in diameter, then the aggregates may be engulfed by macrophages that carry the aggregates into the membrane. However, when the aggregates of the present disclosure exceed about 20, or about 30 microns in diameter, then the aggregates are not readily engulfed by macrophages but instead may directly bind to the membrane by electrostatic interactions. Thus, in one aspect, the present disclosure provides aggregates that bind directly to membrane, such as synovial membrane, and then provide a depot for the release of pharmaceutically active agent from the aggregates as those aggregates are degraded by, e.g., lysozymes.

In one embodiment, the present disclosure provides a composition comprising particles, the particles having a net positive charge, at least some of the particles being spherical with a diameter within the range of at least 10 or 15 or 20 or 25 or 30 microns to 120 or 110 or 105 or 100. Optionally, the particles are sufficiently large in diameter that they do not undergo phagocytosis by macrophages when the particles are administered to synovial fluid. Optionally, the particles become embedded in a nearby synovium when the particles are administered to synovial fluid. The particles may incorporate an active pharmaceutical ingredient (API), where the particles gradually release the API when the particles are administered to synovial fluid.

Thus in one aspect the present disclosure provides a depot for the delivery of pharmaceutical agent from tissue associated with a joint space. Such tissue may be, for example, a membrane such as a synovial membrane, which is also referred to herein as a synovium. The depot serves to store pharmaceutical agent, such as a crystalline form of pharmaceutical agent, and to release pharmaceutical agent to the joint area. The depot provides an extended release of pharmaceutical agent, where that release may be characterized in terms of the number of days during which pharmaceutical agent is released from the aggregates. The number of days may be selected from 1, or 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 41, or 42, or 43, or 44, or 45, or 46, or 47, or 48, or 49, or 50, or 51, or 52, or 53, or 54, or 55, or 56, or 57, or 58, or 59, or 60, for example. For clarity, it should be noted that when the aggregates are characterized in terms of having a release time of 9 days, that means that some pharmaceutical agent is released from the aggregates on each of days 1-9, where day 1 is the day that the aggregate is injected into the host. Additional release may occur. For example, additional release may occur on day 10, or day 11, etc. Thus, a release time of 9 days includes a release time of, for example, 10 days. However, a release time of 10 days does not include a release time of 9 days unless some release of active agent occurs on day 10.

The amount of pharmaceutical agent that is released from the depot, and thus delivered locally to the surrounding tissue and/or joint space, may be characterized in terms of mg of pharmaceutical agent, or in terms of the percent of pharmaceutical agent initially present in the aggregates, as two options. In various embodiments, 0.5 mg, or 0.6 mg, or 0.7 mg, or 0.8 mg, or 0.9 mg, 1.0 mg, or 1.1 mg, 1.2 mg, or 1.3 mg, or 1.4 mg, or 1.5 mg, or 1.6 mg, or 1.7 mg, or 1.8 mg, or 1.9 mg, or 2.0 mg, or 2.1 mg, or 2.2 mg, or 2.3 mg, or 2.4 mg, or 2.5 mg, or 2.6 mg, or 2.7 mg, or 2.8 mg, or 2.9 mg, or 3.0 mg, or 3.2 mg, or 3.4 mg, or 3.6 mg, or 3.8 mg, or 4.0 mg, or 4.2 mg, or 4.4 mg, or 4.6 mg, or 4.8 mg, or 5.0 mg, or 5.2 mg, or 5.4 mg, or 5.6 mg, or 5.8 mg, or 6.0 mg, or 6.2 mg, or 6.4 mg, or 6.6 mg, or 6.8 mg, or 7.0 mg, or 7.2 mg, or 7.4 mg, or 7.6 mg, or 7.8 mg, or 8.0 mg, or 8.2 mg, or 8.4 mg, or 8.6 mg, or 8.8 mg. or 9.0 mg, or 9.2 mg, or 9.4 mg, or 9.6 mg, or 9.8 mg, or 10.0 mg or more than 10.0 mg of drug is released from the depot, where the release is optionally further characterized in terms of release during a stated release time period. It should be noted that these release amounts are minimum release amounts, in that a release of, e.g., 1.0 mg occurs so long as at least 1.0 mg of pharmaceutical agent is released. In another embodiment, 5%, or 10%, or 15%, or 20%, or 25%, or 30%, or 35%, or 40%, or 45%, or 50%, or 55%, or 60%, or 65%, or 70%, or 75%, or more than 75% of the pharmaceutical agent is released from the depot, where the release is optionally further characterized in terms of release during a stated release time period. It should be noted that these release percentages are minimum release amounts, in that a release of, e.g., 50% occurs so long as at least 50% of the initial loading of pharmaceutical agent is released from the depot, optionally within a stated time period.

The depot effect is observed upon degradation of the polymer(s) that form the aggregates and surround the crystalline pharmaceutical agent. Upon polymer degradation, chambers that hold the crystalline agent are opened to the fluid and/or tissue that surrounds the aggregates, so that the crystalline agent can escape into the fluid/tissue and then dissolve in the fluid. In one embodiment, chitosan forms some or all of the aggregate polymeric component. Chitosan is susceptible to degradation in tissue and fluids due, at least in part, to the action of the enzyme lysozyme. Lysozyme degrades chitosan to form oligomers. In one embodiment, the rate of chitosan degradation is related to the degree of deacetylation, where chitosan having a higher degree of deacetylation degrades more slowly upon contact with lysozyme. In other words, lysozyme-induced degradation rate of aggregates was dependent on the degree of deacetylation of the chitosan used and decreased with an increase in its deacetylation.

As mentioned above, in addition to, or instead of demonstrating a depot effect, and in addition to, or instead of demonstrating a slow diffusion effect, the aggregates of the present disclosure may demonstrate a burst delivery of pharmaceutical agent. As used herein, a burst delivery refers to a rapid delivery of pharmaceutical agent that occurs immediately upon administration of the composition of the present disclosure to a subject. The pharmaceutical agent that is delivered during this burst phase may originally be either amorphous or crystalline pharmaceutical agent. The burst delivery may be characterized in terms of the amount of pharmaceutical agent that is delivered to the subject, in a form that is free of the aggregates, during a stated period of time. The period of time may be selected from, for example, any of 1, or 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24 hours after administrated of the composition to the subject. The amount of pharmaceutical agent that is delivered during a burst phase may be characterized in terms of the mass of pharmaceutical agent (e.g., in mg), or in terms of the percent of pharmaceutical agent initially present in the aggregates, as two options. In various embodiments, 0.5 mg, or 0.6 mg, or 0.7 mg, or 0.8 mg, or 0.9 mg, 1.0 mg, or 1.1 mg, 1.2 mg, or 1.3 mg, or 1.4 mg, or 1.5 mg, or 1.6 mg, or 1.7 mg, or 1.8 mg, or 1.9 mg, or 2.0 mg, or 2.1 mg, or 2.2 mg, or 2.3 mg, or 2.4 mg, or 2.5 mg, or 2.6 mg, or 2.7 mg, or 2.8 mg, or 2.9 mg, or 3.0 mg, or 3.2 mg, or 3.4 mg, or 3.6 mg, or 3.8 mg, or 4.0 mg, or 4.2 mg, or 4.4 mg, or 4.6 mg, or 4.8 mg, or 5.0 mg, or 5.2 mg, or 5.4 mg, or 5.6 mg, or 5.8 mg, or 6.0 mg, or 6.2 mg, or 6.4 mg, or 6.6 mg, or 6.8 mg, or 7.0 mg, or 7.2 mg, or 7.4 mg, or 7.6 mg, or 7.8 mg, or 8.0 mg, or 8.2 mg, or 8.4 mg, or 8.6 mg, or 8.8 mg. or 9.0 mg, or 9.2 mg, or 9.4 mg, or 9.6 mg, or 9.8 mg, or 10.0 mg or more than 10.0 mg of drug is delivered to the subject during a burst period, in a form that is free from being associated with the aggregates, where the release is optionally further characterized in terms of release during a stated release time period. It should be noted that these release amounts are minimum release amounts, in that a release of, e.g., 1.0 mg occurs so long as at least 1.0 mg of pharmaceutical agent is released. In another embodiment, 5%, or 10%, or 15%, or 20%, or 25%, or 30%, or 35%, or 40%, or 45%, or 50%, or 55%, or 60%, or 65%, or 70%, or 75%, or more than 75% of the pharmaceutical agent is delivered during a burst period, to the subject in a form that is free from being associated with the aggregates, where the release is optionally further characterized in terms of release during a stated release time period. It should be noted that these release percentages are minimum release amounts, in that a release of, e.g., 50% occurs so long as at least 50% of the initial loading of pharmaceutical agent in the composition is released from the composition, optionally within a stated time period.

The amount of agent released during a burst phase, and the kinetics of that release, may be impacted by the properties of the aggregates. For instance, changes to particle size may alter burst amount and timeline of release. In addition, washing or pre-treating the aggregates during manufacturing can alter the burst amount and timeline of release. For example, washing the aggregates with ethanol, PBS buffer, or pure water may decrease the amount of pharmaceutical agent that is available to be released during a burst phase. Other post-manufacturing treatments, such as treatment with sodium (meta) bisulfite or glycine to remove reactive glutaraldehyde crosslinking agent may also impact the amount of pharmaceutical agent that is available to be released during a burst phase.

Various methods may be used to determine aggregate payload. For example, drug-loaded aggregates may be isolated and then a determined weight of isolated aggregates is dissolved in methanol. The methanol solution is analyzed by HPLC or UV analysis, and the results compared against standard concentrations of active agent that are also dissolved in methanol. In this way, the concentration of the drug in the aggregate-derived methanol solution can be determined, and this determination may be used to calculate how much drug was present in the determined weight of aggregates. The initial drug-loaded aggregates may also be characterized by particle size, crosslinking, or other parameters, to allow a determination of the impact of these parameters on aggregate payload. In order to measure a release profile, a solution of aggregates in buffer or pure water may be placed in a shaker bath at room temperature or elevated temperature, and samples are taken periodically and analyzed. For example, 10-20 mg of drug-loaded aggregates may be placed into a container with 20-1,000 ml of PBS buffer, and the container placed into a shaker bath at room temperature or elevated temperature, e.g., 37° C. Samples are taken at various time points including but not limited to: 0.25, 0.5, 1, 3, 8, 24, 48, 168, 336 hrs, and so on in weekly increments. Such samples are analyzed by HPLC or UV analysis.

In one aspect, the aggregate-containing compositions of the present disclosure provide both a burst delivery of pharmaceutical agent, and provide a depot for extended release of pharmaceutical agent, when the composition is administered to a subject in need. In this case, the time period for burst delivery is set for less than 24 hours, and the time period for depot delivery is set to begin after the end of the burst delivery time period, and extending to a stated time of less than 60 days, or less than 90 days, or less than 120 days. For example, for a burst delivery time of 24 hours, in various embodiments the depot effect of the composition may provide pharmaceutical agent for 1, or 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 41, or 42, or 43, or 44, or 45, or 46, or 47, or 48, or 49, or 50, or 51, or 52, or 53, or 54, or 55, or 56, or 57, or 58, or 59, or 60 days after the 24 hr burst period. Overlapping some of this extended release phase may be slow diffusion of amorphous agent from the aggregate.

The amount of pharmaceutical agent that is released from the composition during any one or more of the burst phase, the slow diffusion phase and the depot phase can be described in terms of the total amount of agent in the composition. In various embodiments, the total amount of pharmaceutical agent present in a dose given to a subject may be at least 0.5 mg, or at least 1.0 mg, or at least 1.5 mg, or at least 2.0 mg, or at least, 2.5 mg, or at least 3.0 mg, or at least 3.5 mg, or at least 4.0 mg, or at least 4.5 mg, or at least 5.0 mg, or at least 5.5 mg, or at least 6.0 mg, or at least 6.5 mg, or at least 7.0 mg, or at least 7.5 mg, or at least 8.0 mg, or at least 8.5 mg, or at least 9.0 mg, or at least 9.5 mg, or at least 10.0 mg. However, too much pharmaceutical agent may be pro-inflammatory and so in various embodiments the aggregates, compositions and methods of the present disclosure may be characterized in terms of a maximum amount of pharmaceutical agent present in a dose given to a subject. In various embodiments, that maximum amount may be 20 mg, or 19.5 mg, or 19.0 mg, or 18.5 mg, or 18.0 mg, or 17.5 mg, or 17.0 mg, or 16.5 mg, or 16.0 mg, or 15.5 mg, or 15.0 mg, or 14.5 mg, or 14.0 mg, or 13.5 mg, or 13.0 mg, or 12.5 mg, or 12.0 mg, or 11.5 mg, or 11.0 mg, or 10.5 mg, or 10.0 mg, or 9.5 mg, or 9.0 mg, or 8.5 mg, or 8.0 mg, or 7.5 mg, or 7.0 mg, or 6.5 mg, or 6.0 mg, or 5.5 mg, or 5.0 mg, or 4.5 mg, or 4.0 mg, or 3.5 mg or less than 3.5 mg. The present disclosure thus provides compositions and doses and methods that refers to one or both of a minimum and maximum amount of pharmaceutical agent present in a dose, where those maximum and minimum values may be selected for the foregoing values.

In one aspect, about 20-60% of the pharmaceutical agent in the composition is released during a burst phase that finishes 24 hours after administration, and about 10-40% of the agent is released during an extended release depot phase of 14 days or 30 days or 60 days or 90 days that follows completion of the burst phase.

As mentioned previously, specified particle size, in combination with a net positive charge for the aggregates, is particularly suited for allowing the aggregates to become embedded in the membrane or synovium that surrounds or adjoins a joint space. The selection of chitosan as a component of the aggregates, and the selection of the Degree of Deacetylation (DDA) that characterizes the chitosan, in addition to particle size, impacts the amount of positive charge that is present on the surface of the aggregates. The DDA should be fairly high in order to provide for a relatively large number of positively charged amine groups on the chitosan. In various embodiments, the DDA is greater than 50%, or greater than 55%, or greater than 60%, or greater than 65%, or greater than 70%, or greater than 75%, or greater than 80%, or greater than 85%, or greater than 90%, or greater than 95%. In one embodiment, the DDA is not greater than 95%. In another embodiment, the DDA is not greater than 90%. In yet another embodiment, the DDA is not greater than 85%. Thus, in one embodiment, the present disclosure provides that aggregates are prepared from chitosan having a DDA of between 80 and 85%. Since the pKa of the chitosan amine group is about 6.5, when the aggregates are placed in a biological fluid such as synovial fluid, those amine groups will be protonated and in that form may be referred to as ammonium groups. For convenience, the term amine groups will refer to both the protonated and un-protonated form, unless the context indicates otherwise.

The chitosan DDA should be fairly high to start with because, in one embodiment, the amine groups are consumed by reaction with glutaraldehyde in order to provide crosslinking within the aggregates. The reaction of amine groups with glutaraldehyde will reduce the number of surface amine groups, and accordingly the number of charged amine group present on the surface, all other factors being equal. Since the amount of crosslinking agent used to provide a crosslinked aggregates does not need to be very high, the majority of the amine groups are not involved in the crosslinking reaction, where in various embodiments, at least 95%, or at least 90%, or at least 85%, or at least 80%, or at least 75%, or at least 70%, or at least 65%, or at least 60%, or at least 55%, or at least 50% of the amine groups of chitosan do not participate in a crosslinking reaction.

Aggregates may be characterized by the weight ratio of chitosan (CS) to hyaluronan (HA). Weight ratio may be determined based upon the relatively weights of the components used to make the aggregates. In one aspect, the CS:HA ratio is greater than 1:1, e.g., is greater than 2:1, or greater than 2.5:1, or greater than 3:1, or greater than 3.5:1, or greater than 4:1, or greater than 4.5:1 or greater than 5:1. In other aspects, the CH:HA ratio is less than 1:1, e.g., is less than 1:1.5, or less than 1:2, or less than 1:2.5, or less than 1:3, or less than 1:3.5, or less than 1:4, or less than 1:4.5, or less than 1:5.

The aqueous carrier of the present disclosure is readily formed by combining water with the various other components of the carrier, e.g., salts, which are desirably in dissolved form. These components may be combined and upon stirring the soluble components will readily dissolve in the water.

The aqueous carrier may be a hydrogel. For example, a temperature-sensitive hydrogel such as a chitosan hydrogel which may be prepared by the method of Chen Minyan et al. China Pharm. 21:1991-1993 (2010). This hydrogel is prepared by dissolving chitosan (2% w/v) in 1% aqueous acetic acid, then mixing that solution with glycerol to a volume ratio of 10:1. Borax solution is added to adjust the pH to 6.7. Aggregates according to the present disclosure may then be dispersed in this chitosan hydrogel.

Accordingly, in one embodiment the present disclosure provides a method of delivering a particle into a synovial fluid and a synovium of a subject to provide a depot for sustained release of an active pharmaceutical ingredient (API) from the particle, the method comprising: (a) providing a liquid composition comprising particles, the particles comprising crosslinked chitosan having amino (—NH2) groups, and an active pharmaceutical ingredient (API) incorporated into the particles, the particles having a diameter of greater than 25 microns and less than 100 microns, wherein the crosslinked chitosan is the reaction product of a crosslinking agent and a chitosan having a degree of deacetylation (DDA) of greater than 75% and less than 95%; (b) providing a syringe containing the liquid composition, the syringe having a needle in the range of 18-27 gauge; and (c) intra-articularly injecting the liquid composition through the needle and into a subject in need thereof. Optionally: upon administration, the particles may reside amongst the cells of the synovium of the subject; the particles in the synovium are not contained inside a macrophage when they provide a sustained release of an active pharmaceutical agent, and where the particles release API into the synovium; the API is released from the particles over a period of, e.g., at least 24 hours; the composition delivers a burst of API immediately after the composition is injected into the subject, and thereafter releases the API over an extended period of time of at least 24 hours; after administration and after becoming embedded in the synovium, the particles of the composition releases API over an extended period of time into the synovium; the API is diclofenac, and at least some of the diclofenac incorporated into the particle is in crystalline form.

### Composition Embodiments

As mentioned previously, the present disclosure provides compositions comprising aggregates which may optionally be distributed in a liquid carrier. In one aspect, the aggregates comprise each of hyaluronan, chitosan and a pharmaceutical agent, i.e., each aggregate contains some amount of each of the three named components. In alternative aspects, the composition comprises aggregates that include only one or two of the named components. Each of the disclosed composition may contain pharmaceutical agent that is not in aggregate form, i.e., is in a non-aggregate form, for example, it may be dissolved in the carrier, or suspended in the carrier by way of surfactants or the like.

In one embodiment, a composition of the present disclosure comprises an aggregate of at least three components, where the three required components are hyaluronan, chitosan, and pharmaceutical agent. This aggregate will be referred to as CHDA for convenience, to indicate that it contains chitosan, hyaluronan and drug (pharmaceutical agent) in a single aggregate. The composition containing the CHDA aggregate will be referred to as the CHDA embodiment or CHDA composition.

The relative amounts of those components is conveniently expressed in terms of a weight percent, based on the total weight of all of the component in the aggregates. In one aspect, the hyaluronan is present at a concentration of from 5 to 90 wt %, or from 30 to 85%, or from 50 to 80%, of the total weight of the aggregates. The chitosan is present at a concentration of from 5 to 90 wt %, or from 30 to 85%, or from 50 to 80%, of the total weight of the aggregates. The pharmaceutical agent is present at a concentration of 0.1% to 10%, or from 1% to 8%, or from 2% to 7% of the total weight of the aggregates.

In addition, the CHDA composition may comprise one or more of: chitosan aggregates that do not contain hyaluronan or pharmaceutical agent; hyaluronan aggregates that do not contain chitosan or pharmaceutical agent; pharmaceutical agent aggregates that do not contain chitosan or hyaluranon; an aggregate comprising chitosan and hyaluronic acid but not containing any pharmaceutical agent, an aggregate comprising chitosan and pharmaceutical agent but not containing any hyaluronan, and/or an aggregate containing hyaluronan and pharmaceutical agent but not containing any chitosan. For example, a composition of the present invention comprises CHDA and HA, optionally with additional pharmaceutical agent which may or may not be the same pharmaceutical agent that is a component of the CHDA.

As mentioned previously, although the CHDA contains pharmaceutical agent, the CHDA composition may optionally contain additional pharmaceutical agent (which may or may not be the same as the pharmaceutical agent in the CHDA) in the form of non-aggregate pharmaceutical agent. For example, a composition of the present disclosure may comprise a first drug in the CHDA, so as to be denoted as CHd1A, and a second drug in a non-aggregated form, so as to be denoted as nAd2. The CHDA may contain two drugs, and thus be denoted as CHd1d2A. The composition may comprise two CHDAs, each having a different drug, i.e., a composition comprising CHd1A and CHd2A.

In other embodiments, the present disclosure provides compositions that contain at least one aggregate that comprises two (but not three) components selected from chitosan, hyaluronan and pharmaceutical agent, in addition to a liquid carrier. These compositions may optionally contain second, third or additional aggregates that may likewise contain one or more of (but not all three of) chitosan, hyaluronan and pharmaceutical agent. These compositions will be referred to as CHD compositions for convenience.

As a first embodiment, a CHD composition of the present disclosure may contain an aggregate formed from chitosan and pharmaceutical agent but not containing any hyaluranon. This aggregate will be referred to as chitosan-drug aggregate (denoted CDA for convenience). The concentration of the drug, based on the total weight of the drug combined with the total weight of the chitosan, i.e., on a weight percent basis, is preferably between 0.1% and 30%.

For example, in one embodiment the present disclosure provides a composition comprising: (a) an aqueous media; (b) an NSAID, where the NSAID may be diclofenac or salts thereof, and (c) chitosan particles that are dispersed in the aqueous media, wherein at least one of the following is true: i) the majority of the particles have a diameter in a range of 26-100 microns (optionally 30-80 microns); and/or ii) the chitosan has a degree of deacetylation (DDA) of between 75-95% (optionally 80-92%); and/or iii) the chitosan has a molecular weight of 260-600 kilodaltons.

As a second embodiment, a CHD composition of the present disclosure may contain an aggregate formed from hyaluranon and pharmaceutical agent, but not containing any chitosan. This aggregate will be referred to as hyaluranon-drug aggregate (denoted HDA for convenience). The concentration of the drug, based on the total weight of the drug combined with the total weight of the hyaluronan, i.e., on a weight percent basis, is preferably between 0.1% and 30%.

As a third embodiment, a CHD composition of the present disclosure may contain an aggregate formed from hyaluronan and chitosan, but not containing any pharmaceutical agent. This aggregate will be referred to as chitosan-hyaluronan aggregate (denoted CHA for convenience). The CHA aggregates will contain from 10-90 wt % chitosan and from 90-10% hyaluranon, based on the total weight of chitosan and hyaluranon.

In addition, a CHD composition may comprise one or more additional aggregates not already present in the CHD composition, where these one or more additional aggregates are selected from: chitosan aggregates that do not contain hyaluronan or pharmaceutical agent; hyaluronan aggregates that do not contain chitosan or pharmaceutical agent; pharmaceutical agent aggregates that do not contain chitosan or hyaluranon; an aggregate comprising chitosan and hyaluronic acid but not containing any pharmaceutical agent, an aggregate comprising chitosan and pharmaceutical agent but not containing any hyaluronan, and/or an aggregate containing hyaluronan and pharmaceutical agent but not containing any chitosan.

As mentioned previously, although the CHD compositions may contain pharmaceutical agent as part of an aggregate, the CHD composition may optionally contain additional pharmaceutical agent (which may or may not be the same as the pharmaceutical agent in the aggregate(s) of the CHD composition) in the form of non-aggregate pharmaceutical agent. When the additional pharmaceutical agent is not part of an aggregate, such a composition may be CDA and nAD, e.g., Cd1A and nAd1 when specifically specifying that the aggregate and non-aggregate drugs are identical, or Cd1A and nAd2 when the aggregate and non-aggregate drugs are non-identical. Alternatively, the composition may be HAD and nAD, e.g., Hd1A and nAd1 when specifically specifying that the aggregate and non-aggregate drugs are identical, or Hd1A and nAd2 when the aggregate and non-aggregate drugs are non-identical. As another alternative, the composition may be CHA and nAD.

In addition, a CHD composition may contain two or more different pharmaceutical agents, each in aggregate form. For instance, a single aggregate may contain two or more drugs, e.g., aggregates denoted by Cd1d2A may be in a composition of the present disclosure, and Hd1d2A may be in compositions of the present disclosure, either providing for a composition with an aggregate that contains exactly two pharmaceutical agents. In addition, or alternatively, the two more drugs may be located in separate aggregates within a single composition. For example, a composition comprising Cd1A and Cd2A when chitosan is in combination with d1 and d2 in separate aggregates. Likewise, the present disclosure provides a composition with Hd1A and Hd2A, i.e., a composition having two different hyaluronan aggregates, each carrying a different pharmaceutical agent.

As mentioned previously, the present disclosure provides compositions that comprise one or more distinct aggregates, where an aggregate contains at two or three components selected from hyaluronan, chitosan and pharmaceutical agent. Optionally, the hyaluronan may be present in a distinct aggregate that contains only hyaluronan. Such compositions will be referred to for convenience as HA compositions, and embodiments of the present disclosure that contain HA (hyaluronan-only aggregates) may be referred to as HA embodiments.

While the HA compositions of the present disclosure comprise hyaluronan-only containing aggregates and a liquid carrier, the HA compositions may contain one or more other aggregates. Exemplary additional aggregates are chitosan aggregates that do not contain hyaluronan or pharmaceutical agent; pharmaceutical agent aggregates that do not contain chitosan or hyaluranon; an aggregate comprising chitosan and hyaluronic acid but not containing any pharmaceutical agent, an aggregate comprising chitosan and pharmaceutical agent but not containing any hyaluronan, and/or an aggregate containing hyaluronan and pharmaceutical agent but not containing any chitosan.

Thus the HA compositions may optionally contain pharmaceutical agent as part of an aggregate. However, in another embodiment, the HA compositions contain pharmaceutical agent, which may or may not be the same as the pharmaceutical agent in an aggregate form, in the form of non-aggregate pharmaceutical agent. In one aspect, the HA composition contains two different pharmaceutical agents, optionally both are in aggregate form, and optionally one agent is in aggregate form and the other agent is in non-aggregate form.

As mentioned previously, the present disclosure provides compositions that comprise one or more distinct aggregates, where an aggregate contains at two or three components selected from hyaluronan, chitosan and pharmaceutical agent. Optionally, the chitosan may be present in a distinct aggregate that contains only chitosan. Such compositions will be referred to for convenience as CA compositions, and embodiments of the present disclosure that contain CA (chitosan-only aggregates) may be referred to as CA embodiments.

While the CA compositions of the present disclosure comprise chitosan-only containing aggregates and a liquid carrier, the CA compositions may contain one or more additional aggregates. Exemplary additional aggregates are selected from: hyaluronan aggregates that do not contain chitosan or pharmaceutical agent; pharmaceutical agent aggregates that do not contain chitosan or hyaluranon; an aggregate comprising chitosan and hyaluronic acid but not containing any pharmaceutical agent, an aggregate comprising chitosan and pharmaceutical agent but not containing any hyaluronan, and/or an aggregate containing hyaluronan and pharmaceutical agent but not containing any chitosan.

Thus the CA compositions may optionally contain pharmaceutical agent as part of an aggregate. However, in another embodiment, the CA compositions contain pharmaceutical agent, which may or may not be the same as the pharmaceutical agent in an aggregate form, in the form of non-aggregate pharmaceutical agent. In one aspect, the CA composition contains two different pharmaceutical agents, optionally both are in aggregate form, and optionally one agent is in aggregate form and the other agent is in non-aggregate form.

In each of the composition embodiments disclosed herein, the pharmaceutical agent may be, for example, selected from any of the pharmaceutical agents specifically identified herein. In addition, when a composition contains more than one pharmaceutical agent, the two or more pharmaceutical agents need not be identical. The pharmaceutical composition is preferably in a sterile form.

For example, in one embodiment the present disclosure provides a composition comprising an active pharmaceutical ingredient (API), the composition providing an extended release of the API upon delivery of the composition into the joint. The present disclosure also provides additional embodiments wherein the composition comprising an API is further characterized by any one or more of the following exemplary characteristics: the composition further comprises chitosan; the composition further comprises particles that contain the API; the API is an NSAID; the API is diclofenac, where optionally at least of the diclofenac may be in crystalline form; the composition comprises 1-50 mg of NSAID, e.g., diclofenac; the delivery is by intra-articular injection; the extended release is for a period of at least 24 hours.

As another example, in one embodiment the present disclosure provides a method of providing an extended release of an active pharmaceutical agent (API) to a joint, the method comprising administering a composition comprising the API into the joint, the composition providing an extended release of API over a period of 24 hours or longer. The present disclosure also provides additional embodiments wherein the method of providing an extended release of an active pharmaceutical agent (API) to a joint is further characterized by any one or more of the following exemplary characteristics: the API is an NSAID; the API is diclofenac; the composition comprises 1-50 mg of NSAID; the administration is by intraarticular injection; the composition comprises particles and the API is incorporated into the particles.

The following are some additional exemplary embodiments of the pharmaceutical compositions of the present disclosure:
1) A pharmaceutical composition comprising an aggregate and a liquid carrier, the aggregate comprising chitosan, hyaluronan and pharmaceutical agent.
2) The pharmaceutical composition of embodiment 1 further comprising chitosan aggregates that do not contain hyaluronan or pharmaceutical agent.
3) The pharmaceutical composition of embodiment 1 further comprising hyaluronan aggregates that do not contain chitosan or pharmaceutical agent.
4) The pharmaceutical composition of embodiment 1 further comprising pharmaceutical agent aggregates that do not contain chitosan or hyaluranon.
5) The pharmaceutical composition of embodiment 1 further comprising an aggregate comprising chitosan and hyaluronic acid but not containing any pharmaceutical agent.
6) The pharmaceutical composition of embodiment 1 further comprising an aggregate comprising chitosan and pharmaceutical agent but not containing any hyaluronan.
7) The pharmaceutical composition of embodiment 1 further comprising an aggregate containing hyaluronan and pharmaceutical agent but not containing any chitosan.
8) The pharmaceutical composition of any of embodiments 1-7 further comprising pharmaceutical agent dissolved in liquid carrier.
9) The pharmaceutical composition of any of embodiments 1-8 comprising an analgesic as a pharmaceutical agent in the composition.
10) The pharmaceutical composition of any of embodiments 1-8 comprising an NSAID as a pharmaceutical agent in the composition.
11) The pharmaceutical composition of any of embodiments 1-8 wherein a DMARD is a pharmaceutical agent in the composition.
12) The pharmaceutical composition of any of embodiments 1-8 wherein a DMOAD is a pharmaceutical agent in the composition.
13) A pharmaceutical composition comprising a first aggregate, a second aggregate and a liquid carrier, the first aggregate comprising hyaluronan without chitosan or pharmaceutical agent, and the second aggregate comprising chitosan and pharmaceutical agent without hyaluronan.
14) The pharmaceutical composition of embodiment 13 further comprising pharmaceutical agent dissolved in the liquid carrier.
15) The pharmaceutical composition of embodiment 13 further comprising a third aggregate comprising pharmaceutical agent without chitosan or hyaluronan.
16) The pharmaceutical composition of any of embodiments 13-15 comprising an analgesic as a pharmaceutical agent in the composition.
17) The pharmaceutical composition of any of embodiments 13-15 comprising an NSAID as a pharmaceutical agent in the composition.
18) The pharmaceutical composition of any of embodiments 13-15 wherein a DMARD is a pharmaceutical agent in the composition.
19) The pharmaceutical composition of any of embodiments 13-15 wherein a DMOAD is a pharmaceutical agent in the composition.
20A pharmaceutical composition comprising a first aggregate, the first aggregate comprising chitosan and pharmaceutical agent without hyaluronan.
21) The pharmaceutical composition of embodiment 20 further comprising pharmaceutical agent dissolved in the liquid carrier.
22) The pharmaceutical composition of embodiment 20 further comprising a third aggregate comprising pharmaceutical agent without chitosan or hyaluronan.
23) A pharmaceutical composition comprising a first aggregate, a second aggregate and a liquid carrier, the first aggregate comprising hyaluronan and chitosan without pharmaceutical agent, the second aggregate comprising chitosan and pharmaceutical agent without hyaluronan.
24) The pharmaceutical composition of embodiment 23 further comprising pharmaceutical agent dissolved in the liquid carrier.
25) The pharmaceutical composition of embodiment 23 further comprising a third aggregate comprising pharmaceutical agent without chitosan or hyaluronan.
26) A pharmaceutical composition comprising a first aggregate, a second aggregate and a liquid carrier, the first aggregate comprising hyaluronan and pharmaceutical agent without chitosan, the second aggregate comprising chitosan and pharmaceutical agent without hyaluronan.
27) The pharmaceutical composition of embodiment 26 further comprising pharmaceutical agent dissolved in the liquid carrier.
28) The pharmaceutical composition of embodiment 26 further comprising a third aggregate comprising pharmaceutical agent without chitosan or hyaluronan.
29) A pharmaceutical composition comprising a first aggregate, a second aggregate and a liquid carrier, the first aggregate comprising hyaluronan without pharmaceutical agent or chitosan, the second aggregate comprising chitosan without pharmaceutical agent or hyaluronan.
30) The pharmaceutical composition of embodiment 29 further comprising pharmaceutical agent dissolved in the liquid carrier.
31) The pharmaceutical composition of embodiment 29 further comprising a third aggregate comprising pharmaceutical agent without chitosan or hyaluronan.
32) A pharmaceutical composition comprising a first aggregate and a liquid carrier, the first aggregate comprising hyaluronan without chitosan or pharmaceutical agent.
33) The pharmaceutical composition of embodiment 32 further comprising pharmaceutical agent dissolved in the liquid carrier.
34) The pharmaceutical composition of embodiment 32 further comprising a second aggregate, the second aggregate comprising chitosan and hyaluronan without pharmaceutical agent.
35) The pharmaceutical composition of embodiment 34 further comprising a third aggregate, the third aggregate comprising pharmaceutical agent without chitosan or hyaluronan.
36) The pharmaceutical composition of embodiment 34 further comprising pharmaceutical agent dissolved in the liquid carrier.
37) A pharmaceutical composition comprising a first aggregate and a liquid carrier, the first aggregate comprising chitosan without hyaluronan or pharmaceutical agent.
38) The pharmaceutical composition of embodiment 37 further comprising pharmaceutical agent dissolved in the liquid carrier.
39) The pharmaceutical composition of embodiment 37 further comprising a second aggregate, the second aggregate comprising pharmaceutical agent without chitosan or hyaluronan.
40) A pharmaceutical composition comprising a first aggregate and a liquid carrier, the first aggregate comprising chitosan and hyaluronan without pharmaceutical agent.
41) The pharmaceutical composition of embodiment 40 further comprising pharmaceutical agent dissolved in the liquid carrier.
42) The pharmaceutical composition of embodiment 40 further comprising a second aggregate, the second aggregate comprising pharmaceutical agent without chitosan or hyaluronan.
In each of these exemplary pharmaceutical compositions embodiments 1-42, an analgesic may be present as a pharmaceutical agent. This option is not to be considered limiting on the pharmaceutical compositions of the present disclosure, wherein any of the pharmaceutical agents as disclosed herein may be present as a pharmaceutical agent, and indeed a pharmaceutical agent not specifically disclosed herein may be present in the pharmaceutical composition.

### Composition Preparation

In one aspect, the aggregate(s) is formed separately from the carrier, and then aggregate(s) is combined with the carrier components in order to form a composition of the present disclosure. For example, an aggregate may be prepared by the spray drying or lyophilization techniques described elsewhere herein. The resulting aggregate is then combined with water, or with water containing dissolved components, e.g., salt(s) and/or hyaluronan and/or pharmaceutical agent. Such combinations have good short term stability.

For example, a composition of the present disclosure may be formed in a two-step process as follows. Chitosan aggregates may be prepared by adding TPP solution (3.00 mg TPP/1 mL water) to a solution of chitosan (2.5 mg chitosan/1 mL water) with stirring to a final concentration of chitosan to TPP of 3.0 to 0.5 mg/mL. These aggregates are isolated by centrifugation at 2,000 rpm, washed twice with water and re-dispersed in 1×PBS. Hyaluronic acid (1500 kDa) is added to the suspension to a final concentration of 1% (w/v) and the mixture stirred overnight to dissolve the HA in the water. The product is crosslinked chitosan aggregates in a carrier comprising soluble hyaluronic acid.

Another option that may be used to disperse aggregate(s) in aqueous media is to disperse the aggregate(s) in a relatively volatile organic solvent, then add that solvent/particle combination to water or PBS solution and evaporate the organic solvent. Using this approach, aggregates can be dispersed in water.

As mentioned elsewhere herein, the carrier for the aggregate(s) may include dissolved hyaluronan. A composition comprising aggregate, water and dissolved hyaluronan may be prepared as follows. Aggregate(s) are combined with hexane containing 1% Tween 80 surfactant to a final concentration of about 100 mg/mL. A 200 μL aliquot of this mixture is added to 2 mL of water with intensive stirring to provide a hexane in water emulsion. The emulsion is stirred at 30° C. overnight to achieve complete evaporation of the hexane. A solution of hyaluronan (e.g., Lifecore HA, 700 kDa) at a concentration of 3% (w/v) in 1×PBS or 0.5×PBS was prepared by combining the components and stirring overnight at 100 rpm. Equal weights of the hyaluronan solution and the aggregate suspension are combined and vortexed, following by overnight stirring. This process results in aggregates dispersed throughout an aqueous carrier comprising dissolved hyaluronan, which is one aspect of the present disclosure.

An aqueous solution of hyaluronan will exhibit shear thinning behavior. In general, and keeping all other factors constant, as less hyaluronan is dissolved in the water, the resulting solution tends to display reduced shear thinning behavior. When chitosan aggregates are combined with the aqueous solution of hyaluronan, the combination also shows reduced shear thinning compared to the same solution without the chitosan aggregates. Reduced shear thinning is generally desirable since increases in shear thinning act as an impediment to delivering the composition through a fixed sized syringe needle. Accordingly, the addition of aggregates, e.g., positively charged aggregates, to an aqueous hyaluronan solution can be used to offset an increase in shear thinning that would otherwise occur if that solution is prepared with an increased concentration and/or molecular weight of hyaluronan (both generally desirable for increased residence time of the hyaluronan in a joint space).

### Methods of the Present Disclosure

In one aspect, the present disclosure provides compositions and methods that can be used to place a pharmaceutical agent directly into a distressed joint. Compared to systemic delivery, the local delivery of a pharmaceutical agent minimizes undesirable drug side effects, allows for a reduced dosage of active agent to be delivered to the subject, and enhances the therapeutic level of the agent within the joint where it is needed. Rapid clearance of pharmaceutical agent from the joint has historically been a problem seen when an agent is injected directly into the joint. The present disclosure addresses this problem by combining pharmaceutical agent(s) with aggregates that remain at the site of injection and provide for a sustained release of the drug over time as the aggregate degrades.

The present disclosure provides methods for addressing joint distress in a patient. The joint being treated may be a cartilaginous joint, or the joint may be a synovial joint. More specifically, the joint that is in distress may be, for example, a knee, hip, elbow, wrist, ankle, shoulder, finger or toe joint. The joint may be any one or more of the carpometacarpal (CMC) joints, which are five joints in the wrist.

For example, the present disclosure provides for the treatment of traumatized knees, hips, and other joints diagnosed as suffering from arthritis or other degenerative joint disease such as osteoarthritis, rheumatoid arthritis, or psoriatic arthritis.

Accordingly, in one embodiment the present disclosure provides a method of preventing advancement of inflammation, the method comprising administering to a subject in need thereof an effective amount of particles comprising chitosan and an anti-inflammatory agent, where the subject has experienced a traumatic injury prior to the administration. Any one or more of the following exemplary optional features may further characterize the method of preventing advancement of inflammation: the anti-inflammatory agent is an NSAID; the anti-inflammatory agent is diclofenac, optionally at least in part in crystalline form; and the anti-inflammatory agent is a steroid.

In a related embodiment, the present disclosure provides a method of preventing osteoarthritis, the method comprising administering to a subject in need thereof an effective amount of particles comprising chitosan and an anti-osteoarthritis agent, where the subject has experienced a traumatic injury prior to the administration. Optionally, the anti-osteoarthritis agent is an NSAID; or is a DMOAD; or is a DMARD.

The present disclosure provides methods that create one or more medical benefits that address the problem of joint distress in a subject. For example, in one embodiment the present disclosure provide a method for reducing the pain and/or inflammation in a joint, such as may be caused by arthritis.

In one embodiment, the present disclosure provides compositions and methods that treat joint distress known as arthritis. Arthritis is the condition of having unwanted inflammation in the joint of a subject. In general, the term arthritis refers to more than 100 rheumatic diseases and conditions that affect joints. The two most common types of arthritis are osteoarthritis and rheumatoid arthritis.

Osteoarthritis is the most common type of arthritis, and is seen especially among older people. Osteoarthritis is usually caused by the normal wear and tear that is experienced upon joint usage. Osteoarthritis is sometimes called degenerative joint disease or osteoarthritis. Osteoarthritis mostly affects cartilage, which is the hard, slippery tissue that covers the ends of bones where they come together to form a joint. Healthy cartilage allows bones to glide over one another, and it also absorbs energy from the shock of physical movement. In osteoarthritis, the cartilage breaks down and wears away. This allows bones under the cartilage to rub against one another, causing pain, swelling, and loss of motion of the joint. Over time, the joint may even lose its normal shape. In some instances, small deposits of bone, which are called osteophytes or bone spurs, may grow at the edges of the joint. Bits of bone or cartilage can break off and float inside the joint space, causing still more pain and damage within the joint. Subjects with osteoarthritis usually have joint pain and some movement limitations.

In rheumatoid arthritis, which is the second most common form of arthritis, the immune system attacks the tissues of the joints, leading to pain, inflammation, and eventually joint damage and malformation. Rheumatoid arthritis typically begins at a younger age than osteoarthritis, causing swelling and redness in joints, and may make people feel sick, tired, and uncommonly feverish. Other types of arthritis can be caused by uric acid crystals, infections, and underlying disease states such as psoriasis or lupus.

The present disclosure provides for the treatment of an arthritis when that treatment includes local delivery of a pharmaceutical agent to the distressed joint space or cavity or soft tissue. Non-inflammatory types of arthritis, e.g., osteoarthritis, are usually treated with oral pain medications including NSAIDS, and the present disclosure provides for the local delivery of pain medication as a pharmaceutical agent. Inflammatory types of arthritis, e.g., rheumatoid arthritis, may also be treated with oral pain medication, but in addition may be treated with oral or systematic injectable anti-inflammatory pharmaceutical agents, e.g., steroids such as corticosteroids. In addition, inflammatory types of arthritis may be treated with disease-modifying anti-rheumatic drugs (DMARDs) or biologics against anti-inflammatory targets. The present disclosure provides for the inclusion of medication(s) for non-inflammatory and inflammatory arthritis as a pharmaceutical agent for local delivery in the compositions of the disclosure.

Thus, in one embodiment, the present disclosure provides both methods that reduce the inflammation in a joint, and compositions that may be used to reduce the inflammation in a joint. That inflammation may be caused by arthritis as discussed above, but it may alternatively be due to other causes, e.g., an alternative disease state or trauma to the joint. For example, in one embodiment the present disclosure provide a method for treating frozen shoulder, also known as adhesive capsulitis. The method includes administering an aggregate as disclosed herein directly to the shoulder of a subject who is suffering from frozen shoulder. For example, the administration may be to the shoulder capsule, the rotator cuff tendon, the shoulder joint, soft tissue in the vicinity of the shoulder joint, or the synovial fluid that surrounds the shoulder capsule and the shoulder joint. The administration may be by injection, where the injection delivers a therapeutically effective amount of a pharmaceutical agent such as an NSAID, e.g., diclofenac. The method of the present disclosure alleviates the pain and stiffness that characterizes frozen shoulder. In a subject who has severe frozen shoulder, where shoulder movement is significantly curtailed, the method of the present invention provides greater ease and range of movement of the shoulder. The method of the present invention may reduce the inflammation that is present in the smooth tissue of the shoulder capsule of a subject who is suffering from frozen shoulder.

In another embodiment, the patient has joint distress in the form of pain that occurs during joint movement, but the cause of the pain is not due to arthritis, and may in fact not have a known cause. Thus, in one embodiment, the subject is suffering from chronic joint pain. The subject having chronic joint pain may or may not have been diagnosed with a joint disease. In another embodiment, the joint pain is asymptotic joint pain, and in another embodiment the pain is idiopathic joint pain. In these embodiments, the pain occurs in a traumatic, non-diseased joint where no clinical diagnosis of cartilage damage, degeneration or trauma, such as osteoarthritis, has been made. Thus, the present disclosure provides for the local delivery of a pain medication to the joint of a subject in need thereof, to reduce pain associated with movement of the joint.

Administration of the compositions of the present disclosure provide medical benefits and health improvements for the patients receiving such compositions. For example, the compositions may provide enhanced lubrication and/or cushioning of a joint, which is particularly desirable in a diseased or otherwise distressed joint in order to assist in improving or restoring proper joint function. The enhanced joint lubrication will typically reduce pain associated with joint movement, where that pain reduction may be supplemented and/or extended by delivery of analgesic agent(s) present in the aggregates or elsewhere in the compositions. For example, the chitosan present in the compositions may provide an analgesic effect on inflammatory pain within the joint space (see, e.g., Okamoto Y., et al., Carbohydrate Polymers 49:249-252 (2002)).

The enhanced lubrication and/or cushioning of a joint, as well as the other benefits immediately or quickly afforded to the subject by administrating a composition of a present disclosure to a subject, can be used to delay more serious treatments and modalities. For example, a patient who might otherwise need to have a knee replacement in the short term, may instead be treated by a composition of the present disclosure in order to maintain the status quo of the joint and thereby delay the need for surgical intervention, e.g., a knee replacement or other joint replacement, to address the problem. In other words, the enhanced mobility and reduced pain that may be achieved by administering the compositions of the present disclosure may benefit the subject by delaying the need for more invasive and risky procedures.

The formulations of the present disclosure may be used to pretreat in the case of traumatic injury OA (such as may occur with people engaged in military action). Abnormal mechanical stresses such as may occur during military action may lead to articular cartilage degeneration in osteoarthritis. Such activity may cause chondrocyte apoptosis and metabolic derangement. Oxidative damage may be the immediate cause of these harmful effects and so antioxidant defenses of chondrocytes may provide tolerance for mechanical injury. Antioxidant defenses in many cell types are stimulated by moderate oxidant exposure. Accordingly, in one aspect the present disclosure provides for oxidant preconditioning in order to reduce acute chondrocyte death and proteoglycan depletion in cartilage explants after exposure to abnormal mechanical stresses.

The formulations of the present disclosure may have a beneficial effect on post traumatic OA. Formulations that contain anti-oxidants (including NSAIDs, e.g., diclofenac) may be applied prior to, e.g., within hours of, injury in order to prevent progressive of mechanically-induced chondrocyte damage and matrix degradation. In addition, pre-treatment may provide for an increase in synovial superoxide activity. The compositions of the present disclosure that include a pharmaceutical agent that reduces superoxide dismutase activity may therefore be beneficial in post-traumatic treatments regimens. The NSAIDs may also modulate the activity of inflammatory cells thereby reducing the release of oxidative metabolites. The present disclosure provides methods for achieving this beneficial result, which comprise delivery formulations as disclosed herein to the joint space. The local delivery to the joint space provides for a high concentration of antioxidant in the space, with a corresponding high effective activity.

A subject treated by the methods and compositions of the present disclosure may have joint distress. As used herein, joint distress refers to one or more of pain in the joint, inflammation in the joint, trauma in the joint, disease in the joint, where diseases include deterioration of the joint, for example the normal wear and tear that a joint experiences over time, a damaged joint such as due to a physical injury such as ACL injury, meniscus tear, or cartilage damage or loss. The patient may be of any age.

Thus, in one aspect the present disclosure provides a method for alleviating pain in a joint of a subject, the method comprising administering to the joint of the subject in need thereof an effective amount of a composition, the composition comprising aggregate and carrier, the aggregate comprising chitosan, hyaluronan and pharmaceutical agent. The pharmaceutical agent is an analgesic, for instance, a narcotic analgesic.

In another aspect the present disclosure provides a method for reducing inflammation in a joint of a subject, the method comprising administering to the joint of the subject in need thereof an effective amount of a composition, the composition comprising aggregate and carrier, the aggregate comprising chitosan, hyaluronan and pharmaceutical agent. The pharmaceutical agent may be a NSAID.

In another aspect the present disclosure provides a method for slowing progression of damage in a joint of a subject due to rheumatoid arthritis, the method comprising administering to the joint of the subject in need thereof an effective amount of a composition, the composition comprising aggregate and carrier, the aggregate comprising chitosan, hyaluronan and pharmaceutical agent. The pharmaceutical agent may be a DMARD.

The methods of the present disclosure are not limited to administering a composition to a joint space. The compositions of the present disclosure may be locally administered to a subject at a site not including a joint space, to provide medical benefits and health improvement. As three examples, and as discussed in further detail below, the subject may be suffering from rotator cuff injury, tennis elbow or plantar fasciitis.

For example, in one embodiment the present disclosure provides a method of treatment or prevention comprising administering a composition of the present disclosure to a soft tissue of a subject in need thereof, the composition comprising chitosan and an active pharmaceutical ingredient (API). In optional embodiments, the method may be further characterized by, for example, any one or of the following: the subject has a soft tissue rheumatic syndrome; the subject has a syndrome selected from myofascial pain, carpal tunnel syndrome, stenosing tenosynovitis, and plantar fasciitis; the subject has tennis elbow or golfers elbow; the subject has bursitis; the subject has tendinitis; the soft tissue is selected from tendons, ligaments, bursae and muscles adjacent to a joint. Also optionally, the administration may be by injection. The API may be an NSAID, such a diclofenac as disclosed herein.

A rotator cuff is made up of the muscles and tendons in the shoulder. These muscles and tendons help hold the ball of the upper arm bone firmly in the shoulder socket. The muscles that are positioned around the shoulder joint and comprise the rotator cuff are named supraspinatus, infraspinatus, subscapularis, and teres minor. A rotator cuff injury includes any type of irritation or damage to the rotator cuff muscles or associated tendons. There are three common conditions that can affect the rotator cuff: rotator cuff tendonitis, rotator cuff impingement syndrome and a rotator cuff tear. Causes of a rotator cuff injury may include falling, lifting and repetitive arm activities, especially those done overhead, such as throwing a baseball or placing items on overhead shelves. The present disclosure provides a method for treating a rotator cuff injury, the method comprising administering a composition of the present disclosure to the rotator cuff, for example, to any of the muscles of the rotator cuff, where the composition may optionally include an anti-inflammatory agent and/or an analgesic as an active pharmaceutical agent.

Tennis elbow is a common term for a condition caused by overuse of arm, forearm, and hand muscles that results in elbow pain. This condition is sometimes called lateral epicondylitis or medial epicondylitis. It does not only result from playing tennis, but is more generally caused when the elbow is overworked, usually by repetitive motions of the wrist and arm. Tennis elbow is caused by either abrupt or subtle injury of the muscle and tendon area around the outside of the elbow. Tennis elbow specifically involves the area where the muscles and tendons of the forearm attach to the outside bony area called the lateral epicondyle of the elbow. Overuse injury can affect the back or posterior part of the elbow as well. The present disclosure provides a method for treating tennis elbow, the method comprising administering a composition of the present disclosure to the muscles around the outside of the elbow, for example, to any one or more of the numerous muscles in the area of the elbow, where the composition may optionally include an anti-inflammatory agent and/or an analgesic as an active pharmaceutical agent.

The plantar fascia is the long, thin ligament that lies directly beneath the skin on the bottom of a person's foot. It connects the heel to the front of the foot, and supports the arch of the foot. The plantar fascia absorbs the high stresses and strains that are placed on the foot during walking, running and other movement. Sometimes, too much pressure damages or tears the tissues, leading to local inflammation, which results in the heel pain and stiffness of plantar fasciitis. The pain may also be caused by collagen degeneration at the origin of the plantar fascia at the medial tubercle of the calcaneus. This degeneration is similar to the chronic necrosis of tendonosis, which features loss of collagen continuity, increases in ground substance (matrix of connective tissue) and vascularity, and the presence of fibro-blasts rather than the inflammatory cells usually seen with the acute inflammation of tendonitis. The cause of the degeneration is repetitive microtears of the plantar fascia that overcome the body's ability to repair itself. Plantar fasciitis is the most common cause of pain on the bottom of the heel. The classic sign of plantar fasciitis is that the worst pain occurs with the first few steps in the morning, but not every patient will have this symptom. Approximately 2 million patients are treated for this condition every year in the United States. The present disclosure provides a method for treating injury and/or degeneration at the plantar fascia, the method comprising administering a composition of the present disclosure to the area of the plantar fascia, where the composition may optionally include an anti-inflammatory agent and/or an analgesic as an active pharmaceutical agent.

Thus, in exemplary embodiments, the present disclosure provides methods of treatment as follows, where these methods are exemplary only and non-limiting on the scope of the present disclosure:
1) A method for alleviating pain in a joint of a subject, the method comprising administering to the joint of the subject in need thereof an effective amount of a pharmaceutical composition of any of exemplary embodiments 1-42.
2) A method for reducing inflammation in a joint of a subject, the method comprising administering to the joint of the subject in need thereof an effective amount of a pharmaceutical composition of any of exemplary embodiments 1-42.
3) A method for slowing progression of damage in a joint of a subject, the method comprising administering to the joint of the subject in need thereof an effective amount of a pharmaceutical composition of any of exemplary embodiments 1-42.
4) A method for reducing inflammation at a site in a subject, comprising administering an effective amount of a pharmaceutical composition of any of exemplary embodiments 1-42 directly to the site of the inflammation.
5) A method for reducing pain at a site in a subject, comprising administering an effective amount of a pharmaceutical composition of any of exemplary embodiments 1-42 directly to the site of the pain.

In each of these exemplary method embodiments 1-5, an analgesic may be used as a pharmaceutical agent. In each of these exemplary method embodiments 1-5, an NSAID may be used as a pharmaceutical agent. In each of these exemplary method embodiments 1-5, a DMARD may be used as a pharmaceutical agent. In each of these exemplary method embodiments 1-5, a DMOAD may be used as a pharmaceutical agent. These four options are not to be considered limiting on the methods of the present disclosure, wherein any of the pharmaceutical agents as disclosed herein may be used as a pharmaceutical agent, and indeed a pharmaceutical agent not specifically disclosed herein may be used in these exemplary methods.

The present compositions provide these benefits with minimal inconvenience to the patient. For example, the delivery of the compositions can typically be achieved in an outpatient setting, during a fairly short period of time. Since the compositions are delivered locally, there is reduced risk of side reactions outside of the joint as might otherwise occur when a therapy is delivered systemically. Minimizing the side effect(s) of a pharmaceutical agent not only makes the therapy safer for a particular patient, but also potentially expands the population that can receive the therapy to include patients whose medical conditions were contraindicated with oral drugs, e.g., diabetics and NSAIDS.

While the methods and compositions of the present disclosure are suitable for an outpatient setting, they are also suitable for use in an in-patient, i.e., hospital or surgical setting. For example, the pharmaceutical compositions may be administered to a subject who is undergoing a surgical procedure, e.g., a joint replacement or the fixing of a meniscus tear. The compositions may also, or alternatively, be administered to a subject in a post-operative setting in order to accelerate the rate of healing.

The methods of the present disclosure are suable for treating people, but they may also be used in veterinary applications. For example, the methods may be used to treat joint distress or soft tissue injury in an animal such as a horse, dog or cat. Likewise, the compositions and dosage forms thereof of the present disclosure may be selected with a view to administration to an animal rather than a person. The terms “subject” and “patient” refer to humans and may optionally be limited thereto. However, more generally, the terms “subject” and “patient” refer to humans and animals that are afflicted with joint distress or soft tissue injury.

The compositions can be administered to the subject in one or more dosage forms over a short or an extended period of time, i.e., over a period of days, weeks, months or even years. In other words, the benefits of the present disclosure may be achieved by a single administration or a series of administrations of the compositions disclosed herein. For example, a subject may receive a weekly administration for 4 weeks, following by a monthly administration for 5 months. Other dosage regimens may be developed based on the severity of the medical condition being treated, the health and other characteristics of the subject receiving the treatment, and the specific aggregates and pharmaceutical agent(s) being administered. The composition of one administration need not be the same as the composition of another administration, which are given to the same subject at different times.

The amount of the API required for use in treatment will vary not only with the particular API selected but also with the route of administration, the nature of the condition being treated and the age and condition of the patient. The dose amount will ultimately be at the discretion of the attendant physician or clinician. However, the effective dose of pharmaceutical agent administered to the subject by a local delivery of aggregates as disclosed herein will typically be significantly less than when that pharmaceutical agent is administered by non-local delivery, e.g., oral administration. For example, and for the average adult human, an intraarticular injection of aggregates that delivers a therapeutically effective amount of diclofenac will have less than 50 mg of diclofenac. The subject will receive such an intraarticular injection periodically, where that period is on the order of weeks or months. In contrast, oral administration of an effective amount of diclofenac is typically on the order to 50-150 mg/day, where this daily dose may be administered for weeks and perhaps months in order to provide efficacy. Accordingly, over a thirty day period, the subject who receives an effective amount of diclofenac by oral administration may be administered on the order of 1500 to 4500 mg of diclofenac, while the subject who receive intraarticular injection according to the present disclosure may receive less than 200 mg of diclofenac in a thirty day period, or less than 100 mg, or less than 50 mg, depending on the dose selected by the attending physician. The local administration and release kinetics of the aggregates of the present disclosure provide an effective amount of NSAID at a much lower dose to the subject compared to a systemic delivery, which is highly advantageous in reducing the side effects which attend the administration of any pharmaceutical agent. Thus, in one embodiment the present disclosure provides for the treatment of osteoarthritis over a 30 days period, and in particular provides for a reduction in the pain associated with osteoarthritis during a 30 day period, through use of less than 50 mg of NSAID, for example, less than 50 mg of diclofenac.

Another advantage of the sustained release formulations of the present disclosure is that the gradual release of drug from the aggregates provides for a more consistent contact between drug and drug receptors. In other words, drug receptor sites are more frequently in contact with drug that is delivered from aggregates of the present disclosure compared to the situation where drug is administered orally in a once or twice a day manner. In the case where the drug is an NSAID, the enhanced frequency of drug-receptor interaction which is realized with the aggregates of the present disclosure will be more effective in having an anti-inflammatory effect. This, in turn, may assist is retarding the progression of osteoarthritis in the subject. In effect, the kinetics of delivery from the aggregates of the present disclosure maintains the drug concentration above a therapeutic threshold concentration that is desirable for retarding OA progression.

The benefits of using intraarticular delivery of drug-bearing aggregates of the present disclosure can also be seen by looking at Drug Targeting Index. The concept of Drug Targeting Index (DTI) is described in the literature, see, e.g., Kirtane A R et al., Pharmaceutical Nanotechnology 2015 104:1174-1186 and Hunt C A et al. Pharm Res 1986 3(6):333-344. The DTI, which quantifies the overall benefit of using a specific encapsulating formulation over free drug, is a ratio of ratios, which can be interpreted as a multiplier for the therapeutic index of the drug due to encapsulation. A large DTI can result from improved targeting to the target compartment (e.g., a joint space), reduced exposure of the toxicity compartment (here the “normal” organs), or both.

In essence, DTI looks at how effectively a molecule of drug interacts with the subject receiving the drug by determining the probably that a given molecule of drug will hit a desired target. With oral delivery, there are many hurdles that a drug such as an NSAID needs to pass in order to be effective, such as safe passage through the gastrointestinal system before reaching the blood stream, and after reaching the blood stream, gaining entry to the knee joint space and particularly the cell receptors in that area which are responsible for inflammatory signals. With intraarticular injection the likelihood that a drug will reach the joint space is greatly improved compared with oral delivery, thus providing a boost to the DTI. In addition, when the aggregates of the present disclosure embed in the synovium they are immediately in the vicinity of the cells that have receptor sites for NSAIDs. Thus, drug that diffuses or otherwise escapes from the aggregates does not have far to travel in order to reach the receptor site. In essence, the concentration of drug and drug receptor is relatively high, immediately upon the drug leaving the aggregate since a relatively small volume is needed in order to include both drug and receptor. Likewise, since the drug is embedded in the area where the drug is targeting, the drug is less likely to reach areas of the subject that can cause unwanted side effects on normal tissue. Drug delivery by the aggregates of the present disclosure therefore provides a relatively large DTI for the drug.

Thus, in one embodiment the present disclosure provides a method for achieving extended release of an active pharmaceutical ingredient (API) into synovial fluid, the method comprising: (a) administering particles to a joint of a subject; (b) allowing the particles to migrate into the synovium; (c) allowing the particles to embed within the synovium; and (d) allowing the particles to degrade in the synovium and release the API into the synovium. In optional embodiments, any one or more of the following exemplary features may further characterize the method for achieving extended release of an API into synovial fluid: the API reduces at least one of pain and inflammation in the subject at the site of administration; the API achieves disease modification; the method increases a Drug Targeting Index of the API for pain and inflammation at the site of administration; the API is an NSAID; the API is diclofenac, where optionally at least some of the diclofenac is in crystalline form.

In a related embodiment, the present disclosure provides a method for raising the Drug Targeting Index (DTI) of an active pharmaceutical ingredient (API), the method comprising administering a composition comprising the API by intra-articular injection to a joint, e.g., the knee. In additional embodiments, any one or more of the following exemplary features may further describe the method for raising the DTI of an API: the API is an NSAID; the API is diclofenac; the DTI is raised relative to a DTI provided by administering the API by any of oral, intravenous (IV), subcutaneously (subQ), topically or intra-muscularly (IM); the composition delivers API to the synovial fluid of the knee; the composition delivers API to the synovium of the knee; API is released from particles embedded in the synovium of the knee; the composition comprises particles, the particles comprising diclofenac and chitosan, at least some of the diclofenac optionally in crystalline form.

The compositions of the present disclosure are also advantageous in terms of the flexibility and versatility of the basic platform. More specifically, the polymeric aggregates as disclosed herein, formed from chitosan and/or hyaluronan, are compatible with a wide range of pharmaceutical agents, including biological materials. The aggregates may be used to encapsulate or otherwise combine with one or more pharmaceutical agents to provide a time-controlled release of agent into the joint space. The polymers from which the aggregates are formed are either naturally occurring or derived from naturally occurring materials that have demonstrated biological safety in multiple settings, e.g., cosmetics and orthopedics.

The compositions of the present disclosure may be in a solid form or a liquid form, and may be delivered to the patient in either of these forms. When in solid form, the composition may be delivered into the patient using, for example, a delivery cannula and dilator assembly as disclosed in, for example, U.S. Pat. Nos. 7,731,981 and 7,914,512. The composition may be in the form of small plugs that will fit inside the cannula, where a suitable size for the plug is on the order of 0.5 mm to 5 mm in diameter. The plug may be porous or nonporous, and it may contain woven or non-woven material to aid in maintaining its shape and consistency. After delivery into the joint space, the composition will absorb moisture from its surroundings and then begin to deliver active agent to the joint.

When in liquid form, the composition may take the form of a paste, gel, solution, suspension, dispersion or the like. It may be delivered to the patient's joint using a hypodermic syringe, i.e., by intra-articular injection. The syringe will have a needle of suitable size, e.g., a needle size on the order of 18-27 gauge, or 18-24 gauge. Alternatively, the fluid composition may be delivered into the joint using a cannula or catheter by methods known to the skilled health care professional.

The in vivo release properties of a candidate formulation may be evaluated in the knee joints of healthy sheep. A formulation is administered by intra-articular injection, with an injection containing either placebo, drug-loaded aggregate, or aggregates that do not contain drug (blank aggregates). For example, a drug-loaded aggregate may be a particle containing chitosan and diclofenac, the particle being suspended in a suitable carrier, e.g., PBS. Various doses and concentrations of drug and polymer (e.g., chitosan) may be evaluated. Synovial fluid and blood may be sampled at various timepoints to evaluate local and systemic pharmokinetics (PK) and pharmodynamics (PD) of the drug, e.g., diclofenac. Optionally, a pro-inflammatory substance such as bacterial lipopolysaccharide may be used to induce a hyperalgesic inflammatory state in the animals, which will be modulated by the formulation of the present disclosure. Fluids may be sampled for as long as needed to establish degradation and residency of the drug-aggregate formulations. The synovial fluid may also be evaluated for the indicators of inflammation including, e.g., leukocyte and protein concentration, optionally over a period of several months. A pharmacodynamics effect will be demonstrated by reduction in synovial fluid inflammation markers, e.g., PGE2, leukocytes, and protein, and/or clinical signs of inflammation as measured by, e.g., effusion, skin temperature and lameness, and/or by restoration of gait parameters as measured by, e.g., ground retention forces using an instrumented walkway. Animals may be evaluated periodically, e.g., daily, for clinical signs of efficacy, e.g., evidence of change in local join irritation. Animals may be weighed at intervals throughout the study. Animals may be euthanized at intermediate time points and at the end of the study, and clinical parameters describing health, appetite and activity levels may be recorded. Joint tissue and regional lymph nodes from each animal may be examined, e.g., spectroscopically, macroscopically, and histologically, for evidence of change in local irritation and for the disposition of the particles. The study may be continued until the active agent (e.g., diclofenac) has cleared from the animal and the aggregate (e.g., chitosan particle) has cleared from the joint.

Candidate formulations may also be evaluated in a peptidoglycan-polysaccharide (PGPS) model of arthritis in rats to evaluate pharmacodynamics. By this study, information about the duration of efficacy in a localized knee synovitis model can be obtained. Initially, synovitis may be induced with an intra-articular injection of PGPS at least a week prior to treatment with a candidate formulation according to the present disclosure. Several doses of a candidate formulation may be evaluated. Subsequent flares of synovitis may be induced by tail vein injections of PGPS during the course of the experiment in order to extend and reactivate the arthritic response. Differences in weight-bearing and gait, which are indicative of knee pain, as well as evaluation of histopathology and pharmacokinetics (PK) may be measured at several time points, over a suitable period, e.g., 4-5 weeks.

The methods and compositions of the present disclosure are illustrated by the following non-limiting example.

### EXAMPLES

### Example 1

### Part 1: Preparation of Chitosan Particles

To 12.5 mL of a 1% v/v solution of aqueous acetic acid was added 187.5 mg of chitosan having a degree of deacetylation of between 83-95% and a molecular weight of 470-500 kilodaltons yielding a 1.5% w/v chitosan solution. Slowly, with continuous mixing, 12.5 mL of acetone was added dropwise to the 1.5% w/v chitosan solution. If precipitation occurs, mix by hand. Separately, 300 mL of cottonseed oil was combined with 2 mL of sorbitane monooleate (SPAN™ 80 surfactant, Croda International PLC; available from, e.g., Sigma-Aldrich, St. Louis, Mo., USA). The 1.5% w/v chitosan solution was added dropwise to the cottonseed oil/SPAN solution with constant stirring to form an emulsion. The container holding the emulsion was placed in a water bath at 37° C., and maintained at that temperature for 14 hours with continuous stirring at 700-800 rpm so that a vortex is present. Separately, 1.2 g of sodium tripolyphosphate (TPP, available from, e.g., Sigma-Aldrich, St. Louis, Mo., USA) was dissolved in 10 mL of water, and then 1 mL of this TPP solution (equivalent to 0.12 g TPP) was added dropwise into the emulsion. The emulsion was then stirred for an additional 4 hours. The emulsion particles were isolated by vacuum filtration using Whatman #4 filter paper. The collected particles were rinsed with equal parts of hexane and then dried overnight to yield 300 mg particles (97.5% yield). The dry particles were observed using an Olympus BX51 light microscope (LEA 08019-01RD) at 200×.

### Part 2: Preparation of Diclofenac-loaded Chitosan Particles

Each of two beakers was charged with 30 mL of 2:1 methanol:water (v:v). The first beaker was additionally charged with 1.00 g sodium diclofenac while the second beaker was additionally charged with 4.16 g sodium diclofenac to provide a saturated sodium diclofenac solution. To each beaker was added 150 mg of the chitosan particles prepared in Part 1 above. The particles were allowed to soak in their respective diclofenac solutions for 24 hours. The particles were isolated by vacuum filtration using Whatman #4 filter paper. The collected particles were rinsed thoroughly with water to remove any residual diclofenac and then allowed to air dry overnight. The dry particles were observed using an Olympus BX51 light microscope (LEA 08019-01RD) at 200×, and looked essentially the same, regardless of the concentration of sodium diclofenac in the methanol:water solutions, with most particles being spherical and having a diameter of between 25 and 100 microns.

The concentration of diclofenac in the particles was determined by extracting diclofenac from the particles through a 25 micron pore size mesh located in 100 mL phosphate buffered saline (PBS) maintained in an incubator/shaker at 37° C. and 60 rpm for 24 hour. By this assay, the particles contained 10-13% by weight diclofenac.

### Example 2

To a suitable vessel was added 7.59 g of chitosan salt (apparent viscosity between 100 and 150 mPa-s; DDA between 83 and 95%) and 700 ml water with stirring to provide a clear solution. This solution was treated with 0.1M NaOH to achieve an acidic solution with pH of 5.4. Then 1633 ml methanol was added to the acidic solution with mixing. Separately, 2.57 diclofenac was dissolved in 26 ml methanol, and this diclofenac solution was added to the chitosan solution with stirring. The mixture of diclofenac and chitosan was placed in contact with a rotor/stator homogenizer (IKA Turex T-25) at 10-15 k rpm while 70 ml of 1% (w/v) TPP in deionized water (pH 9) was added dropwise, for a total addition of 700 mg of TPP to provide a turbid mixture.

A 4-ft diameter stainless steel cone enclosure equipped with a 3-inch diameter toothed spinning disk was used to convert this mixture to particle form, see U.S. Pat. Nos. 7,261,529 and 7,758,778. The air inlet blower speed was 12 Hz, the temperature at the top of the cone was 75-85° C., the temperature at the bottom of the cone was 55-65° C., and the spinning disk speed was adjusted using a hydraulic unit (1.5 gpm) to provide a spin rate of 4600-4800 rpm. The turbid mixture of diclofenac/chitosan/TPP was fed to the center of the spinning disk using a peristaltic pump at 25 ml/min, and 5.2 grams of particles were collected.

A sample of the particle was dissolved in methanol, and the methanol solution was analyzed for the concentration of diclofenac. A concentration (payload) of 23.3% was measured if the particles were not washing with ethanol prior to analysis, and a concentration (payload) of 15.5% was measured in the particles were washed with ethanol prior to analysis. The majority of the particles had a size of 10-40 microns.

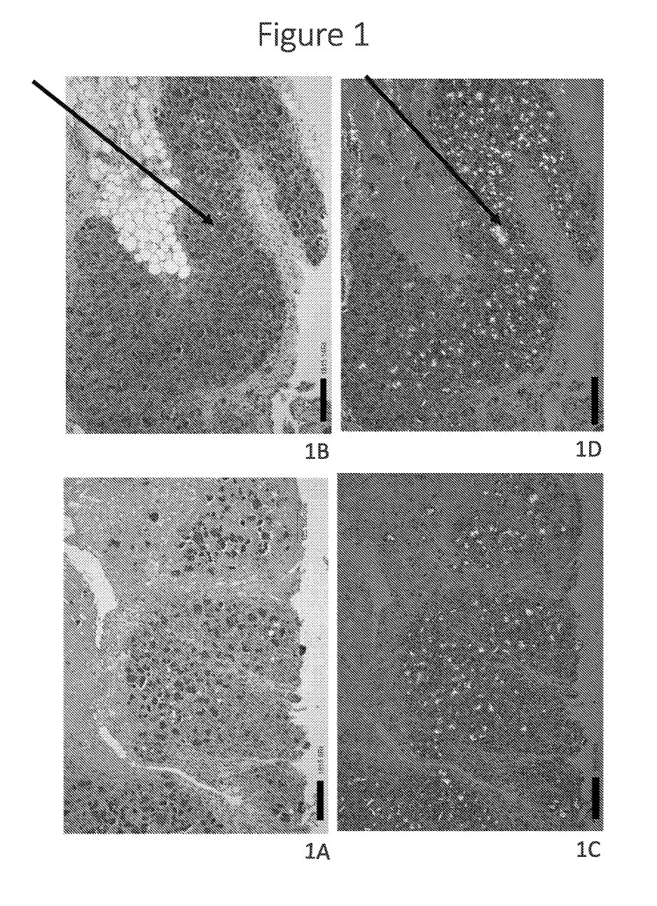

Samples of ethanol-washed particles were injected into sheep knee joints. Sheep were euthanized 7 days and 14 days after injection, and the sheep synovial membrane was examined with the results shown in the Figure. At day 7, microparticles were seen in the synovium, as visualized by adding eosin dye to the membrane, where eosin dye preferentially complexes with the positively charged chitosan amine group (See FIG. 1A). The dark spots in the image (red color in a color micrograph) are the microparticles. At 14 days, particles are still present in the synovium. The black arrow points to one such particle in the day 14 image of FIG. 1B. In addition, the day 7 sample of synovium was examined under fluorescent microscopy. Under these conditions, crystalline material will appear as yellow spots (white spots in a black and white image; see FIG. 1C). Comparison of the Day 7 images as evaluated with eosin dye and with fluorescence microscopy show that the particles and the crystals are in the same locations, indicating that crystals of diclofenac are present in the chitosan particles. This same result is seen in the Day 14 image (see FIG. 1D), where the black arrow points to a white spot that is indicative of diclofenac in crystalline form.

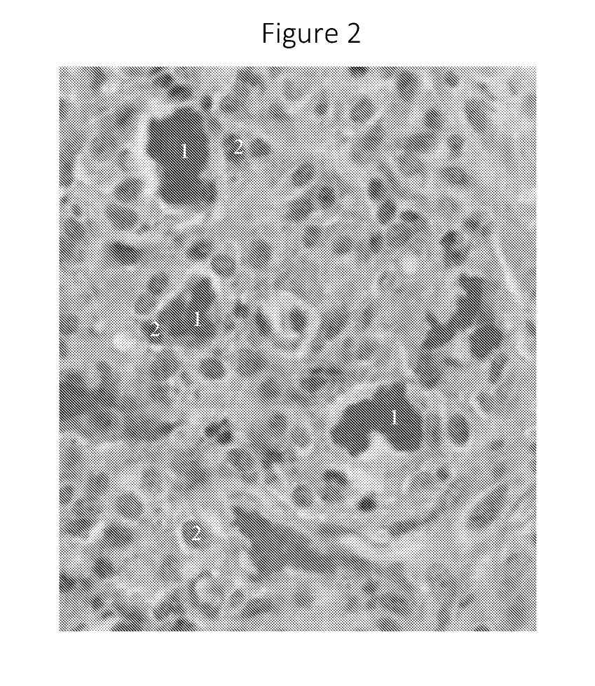

FIG. 2 is another view of the sheep synovium of FIG. 1, where FIG. 2 points out the relative locations of chitosan particles (some of which are labeled “1” in FIG. 2) and synovium macrophages (some of which are labeled “2” in FIG. 2). Each of the particles and the macrophages appear as dark regions when exposed by eosin dye and view microscopically. The particles, e.g., “1”, which have a size on the order of 10-40 microns, are seen to be several times larger than the macrophages, e.g., “2”. By selecting particles that are larger than synovium macrophages, the present disclosure reduces the likelihood that macrophages may engulf the particles by phagocytosis. API that is released from particles that are present within a synovium but which are not engulfed by macrophages is able to immediately disperse throughout the synovium and to exit the synovium and enter the synovial fluid. In other words, the macrophages do not block this release. The API is thus delivered at a higher concentration throughout the joint space when the particles are not engulfed by macrophages, where the macrophages may entrap the API if the particles were located within the macrophages.

### Example 3

The following ingredients, in the order listed and in the amounts stated, were charged to a reaction vessel while maintaining a homogeneous mixture using a rotor/stator homogenizer: 384 g of 1.5% (w/v) chitosan (apparent viscosity between 100 and 150 mPa-s; DDA between 95 and 83%) in water; 1167 ml of methanol; and 2.24 g diclofenac in 22 mL methanol to provide a theoretical drug loading of (2.24/8.97)=25%. Once the mixture appeared clear and homogeneous, there was added 115.5 ml of 1.5% glutaraldehyde (diluted from 25% stock into water) drop-wise while being homogenized at 12-15K rpm. This mixture was allowed to stir for about 15 minutes, and was then used in a spinning disk particle formation process having the following parameters: 4-ft. diameter stainless steel cone enclosure equipped with a 3-in. diameter toothed spinning disk; air inlet blower speed: 10.5 Hz; temperature at top of cone 85-90° C.; temperature at bottom of cone 70-75° C.; spinning disk speed adjusted using hydraulic unit (1.45 gpm) (approximately 4600-4800 rpm). The solution from diclofenac-chitosan/glutaraldehye in methanol/water was fed to the center of the spinning disk using peristaltic pump at 25 mL/min. Collected a total of 4.8 grams of materials (53.5% recovery of total 8.97 grams of solids).

Any of the various embodiments described above can be combined to provide further embodiments. All of the U.S. patents, U.S. patent application publications, U.S. patent applications, foreign patents, foreign patent applications and non-patent publications referred to in this specification are incorporated herein by reference, in their entirety. Aspects of the embodiments can be modified, if necessary to employ concepts of the various patents, applications and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A composition comprising particles, the particles comprising crosslinked chitosan having amino (—NH₂) groups, and an active pharmaceutical ingredient (API) incorporated into the particles, the particles having a diameter of greater than 25 microns and less than 100 microns, wherein the crosslinked chitosan is the reaction product of a crosslinking agent and a chitosan having a degree of deacetylation (DDA) of greater than 75% and less than 95%.

2. The composition of claim 1 wherein the API is an NSAID.

3. The composition of claim 1 wherein the API is diclofenac and at least some of the diclofenac is present in a crystalline form.

4. The composition of claim 1 wherein the crosslinked chitosan is the reaction product of a crosslinking agent that covalently reacts with amino groups.

5. The composition of claim 1 wherein the crosslinked chitosan is the reaction product of glutaraldehyde and chitosan which provides for covalent crosslinks.

6. The composition of claim 1 wherein the crosslinked chitosan is the reaction product of a crosslinking agent and a chitosan having an intrinsic viscosity of greater than 50 mPas and less than 300 mPas.

7. The composition of claim 1 further comprising a liquid medium, the composition being injectable through a needle of 18-27 gauge.

8. The composition of claim 1 wherein the API is diclofenac and at least some of the diclofenac is in crystalline form, the crosslinked chitosan is the reaction product of glutaraldehyde and chitosan which provides for covalent crosslinks, and the crosslinked chitosan is the reaction product of a crosslinking agent and a chitosan having an intrinsic viscosity of greater than 50 mPas and less than 300 mPas.

9. A method of delivering a particle into a synovial fluid and a synovium of a subject to provide a depot for sustained release of an active pharmaceutical agent (API) from the particle, the method comprising:
a. providing a liquid composition comprising particles, the particles comprising crosslinked chitosan having amino (—NH₂) groups, and an active pharmaceutical agent (API) incorporated into the particles, the particles having a diameter of greater than 25 microns and less than 100 microns, wherein the crosslinked chitosan is the reaction product of a crosslinking agent and a chitosan having a degree of deacetylation (DDA) of greater than 75% and less than 95%;
b. providing a syringe containing the liquid composition, the syringe having a needle in the range of 18-27 gauge; and
c. intra-articularly injecting the liquid composition through the needle and into a subject in need thereof.

10. The method of claim 9 to thereby provide for the particles to reside amongst the cells of the synovium of the subject.

11. The method of claim 9 where the particles in the synovium are not contained inside a macrophage when they provide a sustained release of an active pharmaceutical agent, and where the particles release API into the synovium.

12. The method of claim 9 wherein the API is released from the particles over a period of at least 24 hours.

13. The method of claim 9 wherein the composition delivers a burst of API immediately after the composition is injected into the subject, and thereafter releases the API over an extended period of time of at least 24 hours.

14. The method of claim 9 wherein the composition releases API over an extended period of time into the synovium.

15. The method of claim 9 wherein the API is diclofenac, and at least some of the diclofenac incorporated into the particle is in crystalline form.
